(19) United States
(12) Patent Application Publication
(10) Pub. No.: US 2005/0049278 A1
(43) Pub. Date: Mar. 3, 2005

(54) Benzo[1,3]dioxole compounds, pharmaceutical compositions thereof, and processes of making and using the same

(76) Inventors: Youssef Bennani, Shaker Heights, OH (US); James T. Anderson, Shaker Heights, OH (US); Jianmin Wang, Avon, OH (US); Anthony Ting, Shaker Heights, OH (US)

Correspondence Address: FLESHNER & KIM, LLP, P.O. BOX 221200, CHANTILLY, VA, 20153, US

(21) Appl. No.: 10/648,781

(22) Filed: Aug. 27, 2003

(51) Int. Cl. C07D 47/02; A61K 31/4709; A61K 31/404

(52) U.S. Cl. 514/314; 514/414; 546/165; 548/454

(57) ABSTRACT

The present invention relates to benzo[1,3]dioxole compounds and pharmaceutical compositions containing such compounds. These compounds and compositions may be made by various methods. These compounds and compositions may be used to treat disorders and diseases such as cancer.

### BACKGROUND OF THE INVENTION

1. Field of the Invention

The present invention relates to benzo[1,3]dioxole compounds, pharmaceutical compositions containing such compounds, and methods of making and using such compounds and compositions, such as for treatment of cancer.

2. Background

A eukaryotic cell cannot divide into two, the two into four, etc., unless two processes alternate: (a) doubling of its genome (DNA) in S phase (synthesis phase) of the cell cycle; and (b) halving of the doubled genome during mitosis (M phase). The period between M and S is called G₁; that between S and M is G₂. So, the cell cycle consists of: G₁=growth and preparation of the chromosomes for replication; S=synthesis of DNA and centrioles (DNA replications); G₂=preparation for mitosis; and M=mitosis.

The passage of a cell through the cell cycle is controlled by various cytoplasmic proteins. These include, in animal cells, cyclins (G₁ cyclins, S-phase cyclins, and M-phase cyclins) the levels of which in the cell rise and fall with the stages of the cell cycle and with the levels of cyclin-dependent kinases (CDKs) (G₁ CDKs, S-phase CDKs, and M-phase CDKs). CDK levels in the cell remain fairly stable, but each must bind the appropriate cyclin (whose levels fluctuate) in order to be activated (see, e.g., FIG. 1). CDKs add phosphate groups to a variety of protein substrates that control processes in the cell cycle.

A rising level of G₁ cyclins signals the cell to prepare the chromosomes for replication. A rising level of S-phase promoting factor (SPF) prepares the cell to enter S phase and duplicate its DNA (and its centrioles). As DNA replication continues, one of the cyclins shared by G₁ and S-phase CDKs (cyclin E) is destroyed and the level of mitotic cyclins begins to rise (in G₂). The M-phase promoting factor (the complex of mitotic cyclins with M-phase CDK) initiates assembly of the mitotic spindle, breakdown of the nuclear envelope, and condensation of the chromosomes.

These events take the cell to metaphase of mitosis. At this point, the M-phase promoting factor activates the anaphase promoting complex (APC), which activation allows the sister chromatids at the metaphase plate to separate and move to the poles (=anaphase). Completing mitosis destroys the M-phase cyclins. In particular, the M-phase cyclins are conjugated with the protein ubiquitin, which targets them for destruction by proteasomes. Further, the synthesis of G₁ cyclins is turned on for the next turn of the cycle and degrades geminin, a protein that prevents the freshly-synthesized DNA in S phase from being re-replicated before mitosis.

Cell division is dependent, in part, on the formation of a spindle apparatus, which is formed by the polymerization of tubulin. Elements of the spindle apparatus attach to each chromosome and are involved in segregation of a single complement of chromosomes to each of the two daughter cells. When spindle formation is inhibited, the chromosomes do not segregate to the poles of the cell and the cells do not divide. Instead, the cells are blocked at the G2/M boundary and are maintained in this premitotic stage. If cells are maintained in this premitotic stage for a prolonged period of time, they will die from a lack of the normal metabolic activity required to maintain the cells.

Under normal circumstances, the progression through the cell cycle is highly regulated, and errors in the cell progression initiate a sequence of biochemical events leading to programmed cell death or apoptosis. Deregulation of checkpoint mechanisms leads to genetic instability, which is a primary step for tumor development and cancer.

Anticancer therapeutic agents interfere with DNA or RNA structures, interfere with metabolic enzymes, or affect the function of mitotic spindles. Antimitotic drugs affect microtubule dynamics (assembly or stabilization). There are two classes of anti-microtubule agents: (1) those that prevent the assembly of tubulin (e.g., colchicines, podophyllotoxin, MTC [2-methoxy-5-(2,3,4-trimethoxyphenyl)-2,4,6-cycloheptatien-1-one], and vinca alkaloids (e.g., vinblastin, vincristin)); and (2) those that promote the assembly of microtubules (e.g., paclitaxel, taxotere, epothilone A and B, and derivatives thereof).

Noscapine is a naturally occurring isoquinoline alkaloid obtained from opium. Animal studies have shown that its use as a cough suppressant is equivalent to codeine. YE et al., PNAS, 95:1601-1606 (1998), discloses that noscapine arrests mammalian cells at mitosis (M-phase) and causes apoptosis in cycling cells. See also, e.g., WO 99/08528; U.S. Pat. No. 6,376,516 to JOSHI et al.; and AGGARWAL et al., Rapid Commun. Mass Spectrom., 16:923-928 (2002).

Noscapine exists in abundance in the opium plant Papaver somniferum L. papaveraceae. It is commercially available at low cost and in large quantities. SCHMIDHAMMER et al., Arch. Pharm., 311:664-671 (1978), discloses that noscapine may undergo nucleophilic substitution of the 7-methoxy group with alkoxides under anhydrous conditions, with concomitant isomerization in the 3-position. SCHMIDHAMMER discloses that 7-OH, 7-O-ethyl, 7-O -butyl, 7-O-[2″-diethylamino]-ethyl, and 7-O-t-butyl noscapine derivatives are antitussives.

NAKAGAWA et al., Japan J. Pharmacol., 45:417-424 (1987), discloses that tritoqualine inhibits interleukin stimulated cell proliferation. NAKAGAWA also discloses that the effects of tritoqualine on various tumor cell lines that proliferate without interleukin were investigated to see whether the inhibitory effect might be specific for interleukin-induced proliferation. NAKAGAWA reports that tritoqualine inhibited tumor cell proliferation; however, only at a higher concentration.

EP 0 201 359 discloses the use of phthalide isoquinoline derivatives for treatments inhibiting the formation of lipid peroxides.

MARTON et al., Monatshefte fur Chemie Chemical Monthly, 124:291-297 (1993), discloses that some synthetic aminophthalideisoquinolines have anti-allergic activity or hepatoprotective activity. MARTON discloses that the nitro- and amino-phthalideisoquinolines exist as two diastereomeric pairs of enantiomers and two racemates.

U.S. Pat. No. 4,704,458 to TAKEDA et al. discloses processes for the epimerization of aminated phthalideisoquinolines that may be used to treat liver or allergy diseases.

U.S. Pat. No. 4,684,732 to TANIGUCHI et al. discloses processes for preparation of phthalide derivatives that may be used as antiallergy agents.

GB 873935 discloses improvements in and relating to new isoquinoline phthalides and their process of preparation. GB 873935 discloses that these compounds may be useful in preserving food and treating allergies or nausea.

HU 207 324 discloses preparation of aminophthalide isoquinolines that may be used as antiallergy or hepatoprotective agents. This document discloses that both a 1RS-3′RS (A-mer) and a 1RS-3′SR (B-mer) epimer are formed in the course of condensation. This document also discloses that the purpose of its epimerization is to obtain the pharmacologically active A-mer from a mixture of A-mer and B-mer.

There remains, however, a need for new benzo[1,3]dioxole compounds and for pharmaceutical compositions containing such compounds. Accordingly, there remains a need for making these compounds and compositions. There also remains a need for new methods of using these compounds, such as for treating diseases like cancer.

### SUMMARY OF THE INVENTION

Accordingly, the present invention provides benzo[1,3]dioxole compounds, such as noscapine derivatives, and pharmaceutical compositions containing these compounds. The present invention also provides methods of making and using these compounds and compositions. For instance, the present invention provides methods of treating cancer by administering an effective amount of one or more benzo[1,3]dioxole compounds, such as noscapine derivatives, and/or pharmaceutical compositions containing these compounds.

A first embodiment of the present invention is therefore directed to a compound of formula (I): 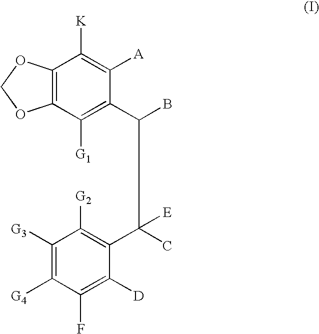
wherein:
A is (i) 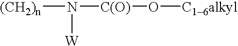
   in which W is C₁₋₆alkyl or C₁₋₆alkylaryl and n=0, 1, or 2, or
(ii) 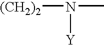
   and forms a nitrogen-containing heterocycloalkyl ring with
B,
in which Y is:
   (a) hydrogen, C₁₋₆alkyl, or C₁₋₆alkylaryl,
   (b) —C(O)—C₁₋₆alkyl or —C(O)—C₁₋₆alkylaryl,
   (c) —CH₂—CH(OH)—CH₂-Z, where Z is C₁₋₆alkyl or —O—C₁₋₆alkyl,
   (d) aryl, or
   (e) heteroaryl;
B is —OH, halogen, or a single bond that forms a six-membered heterocycloalkyl ring with A;
C is hydrogen, C₁₋₆alkyl, or halogen;
D is (i) —CH₂-halogen, —CH(O), —COOH, —C(O)—O—C₁₋₆alkyl, —C(O)—O—C₁₋₆alkylaryl, —CH₂OH, or —(CH₂),—CH₃, wherein n is 1, 2, or 3, or
   (ii) together with E forms a five- or six-membered cycloalkyl or heterocycloalkyl ring;
E is —OH or C₁₋₆alkyl, or together with D forms a five- or six-membered cycloalkyl or heterocycloalkyl ring, wherein this heterocycloalkyl ring contains —C(O)O—, —C(O)NH—, —C(S)O—, or —C(S)NH—;
F is hydrogen, —O—C₁₋₆alkyl, —O—C₁₋₆alkylaryl, —O—C₁₋₆alkylheteroaryl, halogen, aryl, C₁₋₆alkyl, —SH, thio-C₁₋₆alkyl, —S-aryl, —O—SO₂—C₁₋₆alkyl, —O—SO₂—C₁₋₆alkylaryl, cyano, or NR1R2, where R1 and R2 are independently hydrogen, C₁₋₆alkyl, C₁₋₆alkylaryl, cyano, aryl, heteroaryl, —SO₂—C₁₋₆alkyl, or —SO₂—N(C₁₋₆alkyl)(C₁₋₆alkyl);
G₁ to G₄ independently represent hydrogen, aryl, halogen, C₁₋₆alkyl, hydroxyl, —S—C₁₋₆alkyl, nitro, —O—C₁₋₆alkyl, —O—C₁₋₆alkylaryl, or —(CH₂)ₓNR1R2, where x is 0, 1, or 2 and where R1 and R2 are independently hydrogen, C₁₋₆alkyl, C₁₋₆alkylaryl, cyano, aryl, heteroaryl, or acyl, or
two adjacent G₂ to G₄ groups together comprise an alkylene —(CH₂)ₘ—, where m is 3 or 4, to form a cycloalkyl ring, or together comprise an alkylene dioxy —O—(CH₂)ₙ—O—, where n is 1, 2, or 3, to form a heterocycloalkyl ring; and
K is C₁₋₆alkyl, halogen, cyano, aryl, hydrogen, hydroxyl, thio-C₁₋₆alkyl, sulfonyl, sulfoxyl, nitro, —O—C₁₋₆alkyl, —O—C₁₋₆alkylaryl, or NR1R2, where R1 and R2 are independently hydrogen, C₁₋₆alkyl, C₁₋₆alkylaryl, cyano, aryl, heteroaryl, or acyl;
wherein one or more of said alkyl, aryl, heteroaryl, cycloalkyl, heterocycloalkyl, and alkylaryl groups are optionally substituted with one or more suitable substituents;
   a salt thereof, a solvate thereof, a solvated salt thereof, or a combination of two
or more thereof;
provided that when A is —(CH₂)₂—N(Y)— and forms a nitrogen-containing heterocycloalkyl ring with B, and D together with E forms an unsubstituted five-membered heterocycloalkyl ring that contains —C(O)O—, then:
   (i) F is not unsubstituted —O—C₁₋₆alkyl or dialkylamino-substituted —O—C₁₋₆alkyl when G₁ is hydrogen, hydroxyl, or unsubstituted —O—C₁₋₆alkyl, G₂ is hydrogen, halogen, or a nitrogen-containing radical, G₃ is hydrogen, G₄ is hydroxyl or unsubstituted —O—C₁₋₆alkyl, and Y is hydrogen, unsubstituted C₁₋₆alkyl, oxo-substituted C₁₋₆alkyl, thiocarbamoyl-substituted C₁₋₆alkyl, hydroxy-substituted C₁₋₆alkyl, or heteroaryl,
   (ii) F is not —NO₂ or NR1R2 where R1 and R2 are both hydrogen or the same oxo-substituted C₁₋₆alkyl (a) when at least three of G₁, G₂, G₃, and G₄ are the same unsubstituted —O—C₁₋₆alkyl or (b) when G₂ is —NO₂, and
   (iii) F is not hydrogen (a) when G₂, G₃, and G₄ are all hydrogen or (b) when G₂ and G₃ or G₃ and G₄ together comprise a methylenedioxy or (c) when at least two of G₂, G₃, and G₄ are unsubstituted —O—C₁₋₆alkyl or (d) when G₁ is unsubstituted —O—C₁₋₆alkyl and G₄ is a nitrogen-containing radical or halogen.

In another aspect, the present invention is directed to a synthetic method, comprising converting a compound of formula (II): 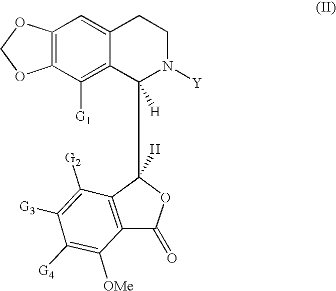
wherein:
Y is:
   (a) hydrogen, C₁₋₆alkyl, or C₁₋₆alkylaryl,
   (b) —C(O)—C₁₋₆alkyl or —C(O)—C₁₋₆alkylaryl,
   (c) —CH₂—CH(OH)—CH₂-Z, where Z is C₁₋₆alkyl or —O—C₁₋₆alkyl,
   (d) aryl, or
   (e) heteroaryl; and
G₁ to G₄ independently represent hydrogen, aryl, halogen, C₁₋₆alkyl, hydroxyl, —S—C₁₋₆alkyl, nitro, —O—C₁₋₆alkyl, —O—C₁₋₆alkylaryl, or —(CH₂)ₓNR1R2, where x is 0, 1, or 2 and where R1 and R2 are independently hydrogen, C₁₋₆alkyl, C₁₋₆alkylaryl, cyano, aryl, heteroaryl, or acyl, or
two adjacent G₂ to G₄ groups together comprise an alkylene —(CH₂)ₘ—, where m is 3 or 4, to form a cycloalkyl ring, or together comprise an alkylene dioxy —O—(CH₂)ₙ—O—, where n is 1, 2, or 3, to form a heterocycloalkyl ring;
a salt thereof, a solvate thereof, a solvated salt thereof, or a combination of two or more thereof;
into a single stereoisomer of formula (III): 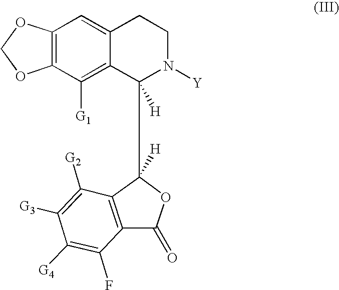
   wherein G₁, G₂, G₃, G₄, and Y are as defined above, and F is —O—C₂₋₆alkyl, —O—C₁₋₆alkylaryl, —O—C₁₋₆alkylheteroaryl, halogen, aryl, C₁₋₆alkyl, —SH, thio-C₁₋₆alkyl, —S-aryl, —O—SO₂—C₁₋₆alkyl, —O—SO₂—C₁₋₆alkylaryl, cyano, or NR1R2, where R1 and R2 are independently hydrogen, C₁₋₆alkyl, C₁₋₆alkylaryl, cyano, aryl, heteroaryl, —SO₂—C₁₋₆alkyl, or —SO₂—N(C₁₋₆alkyl)(C₁₋₆alkyl), provided that F is not —O-t-C₄H₉ or —O—CH₂CH₂N(C₂H₅)₂; wherein one or more of said alkyl, aryl, heteroaryl, and alkylaryl groups are optionally substituted with one or more suitable substituents; a salt thereof, a solvate thereof, a solvated salt thereof, or a combination of two or more thereof.

Another aspect of the present invention is directed to pharmaceutical compositions containing an effective amount of a compound of formula (I) and a pharmaceutically acceptable carrier.

Still another aspect of the present invention is directed to a method of treating cancer by administering to a mammal in need thereof an effective amount of a compound of formula (I), a pharmaceutically acceptable salt thereof, a pharmaceutically acceptable solvate thereof, a pharmaceutically acceptable prodrug thereof, a pharmaceutically acceptable solvated salt thereof, a pharmaceutically acceptable solvated prodrug thereof, a pharmaceutically acceptable salt of a prodrug thereof, or a combination of two or more thereof, provided that when A is —(CH₂)₂—N(Y)—and forms a nitrogen-containing heterocycloalkyl ring with B, and D together with E forms an unsubstituted five-membered heterocycloalkyl ring that contains —C(O)O— then:
(i) F is not unsubstituted —O—C₁₋₆alkyl when G₁ and G₄ are the same unsubstitued —O—C₁₋₆alkyl and Y is unsubstituted C₁₋₆alkyl, carbamoyl-substituted C₁₋₆alkyl, thiocarbamoyl-substituted C₁₋₆alkyl, hydroxy-substituted C₁₋₆alkyl, or heteroaryl, and
(ii) F is not unsubstituted —O—C₁₋₆alkyl when G₁ is unsubstituted —O—C₁₋₆alkyl, G₄ is hydroxyl, and Y is unsubstituted C₁₋₆alkyl.

In another aspect, the present invention is directed to a method of inhibiting mitotic spindle formation, comprising contacting a cell, e.g., a unicellular organism or at least one cell of a multicellular organism, with an effective amount of a compound of formula (I).

In still another aspect, the present invention is directed to a method of inhibiting mitosis, comprising contacting a cell with an effective amount of a compound of formula (I).

In yet another aspect, the present invention is directed to a method of inducing apoptosis, comprising contacting a cell with an effective amount of a compound of formula (I).

In a further aspect, the present invention is directed to a method of inhibiting the cell cycle, comprising contacting a cell with an effective amount of a compound of formula (I).

In another aspect, the present invention is directed to a method of inhibiting cell division, comprising contacting a cell with an effective amount of a compound of formula (I).

In still another aspect, the present invention is directed to a method of arresting cells in S-phase, comprising contacting a cell with an effective amount of a compound of formula (I).

In yet another aspect, the present invention is directed to a method of arresting cells in G2/M, comprising contacting a cell with an effective amount of a compound of formula (I).

In a further aspect, the present invention is directed to a method of inhibiting topoisomerase I, comprising contacting topoisomerase I with an effective amount of a compound of formula (I).

In another aspect, the present invention is directed to a method of inhibiting topoisomerase II, comprising contacting topoisomerase II with an effective amount of a compound of formula (I).

In a further aspect, the present invention is directed to a method of inhibiting microtubule polymerization, comprising contacting a cell with an effective amount of a compound of formula (I).

In another aspect, the present invention is directed to a method of inhibiting yeast growth, comprising contacting a yeast with an effective amount of a compound of formula (I).

In still another aspect, the present invention is directed to a method of inhibiting fungal growth, comprising contacting a fungus with an effective amount of a compound of formula (I).

The present invention is also directed to a method of cough suppression, comprising administering to a mammal in need thereof an effective amount of the compound of formula (I).

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

FIG. 1 is a schematic showing the cell cycle.

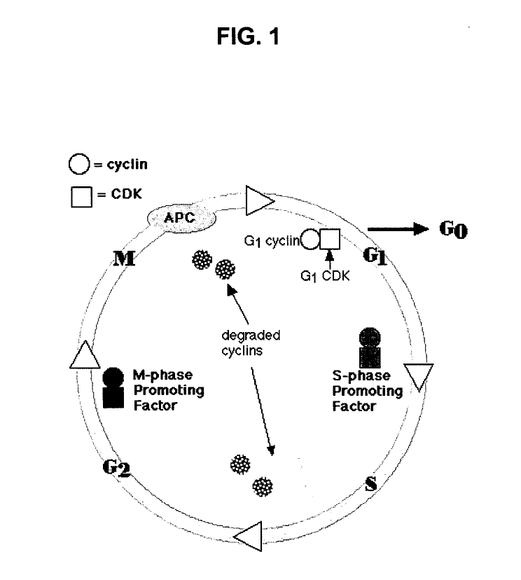

FIG. 2 shows the effect of compounds, including compounds of the present invention, on topoisomerase I and topoisomerase II activity.

FIG. 3 is a graph showing the effect of compounds, including a compound of the present invention, on microtubule polymerization.

### DETAILED DESCRIPTION OF PREFERRED EMBODIMENTS

Definitions

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

As used herein, “alkyl” is intended to mean a straight or branched chain monovalent radical of saturated and/or unsaturated carbon atoms and hydrogen atoms, such as methyl, ethyl, propyl, isopropyl, butyl, isobutyl, t-butyl, ethenyl, pentenyl, butenyl, propenyl, ethynyl, butynyl, propynyl, pentynyl, hexynyl, and the like, which may be unsubstituted (i.e., containing only carbon and hydrogen) or substituted by one or more suitable substituents as defined below.

As used herein, “alkoxy” or “O-alkyl” is intended to mean an oxygen bonded to an alkyl group, wherein the alkyl group is as defined above.

As used herein, “cycloalkyl” is intended to mean a non-aromatic, monovalent monocyclic, bicyclic, or tricyclic radical containing 3, 4, 5, 6, 7, 8, 9, 10, 11, 12, 13, or 14 carbon ring atoms, each of which may be saturated or unsaturated, and which may be unsubstituted or substituted by one or more suitable substituents as defined below, and to which may be fused one or more heterocycloalkyl groups, aryl groups, or heteroaryl groups, which themselves may be unsubstituted or substituted by one or more suitable substituents. Illustrative examples of cycloalkyl groups include, but are not limited to, cyclopropyl, cyclobutyl, cyclopentyl, cyclopentenyl, cyclohexyl, cyclohexenyl, cycloheptyl, cyclooctyl, bicyclo[2.2.1]heptyl, bicyclo[2.2.1]hept-2-en-5-yl, bicyclo[2.2.2]octyl, bicyclo[3.2.1]nonyl, bicyclo[4.3.0]nonyl, bicyclo[4.4.0]decyl, indan-1-yl, indan-2-yl, tetralin-1-yl, tetralin-2-yl, adamantyl, and the like.

As used herein, “heterocycloalkyl” is intended to mean a non-aromatic, monovalent monocyclic, bicyclic, or tricyclic radical, which is saturated or unsaturated, containing 3, 4, 5, 6, 7, 8, 9, 10, 11, 12, 13, 14, 15, 16, 17, or 18 ring atoms, and which includes 1, 2, 3, 4, or 5 heteroatoms selected from nitrogen, oxygen and sulfur, wherein the radical is unsubstituted or substituted by one or more suitable substituents as defined below, and to which may be fused one or more cycloalkyl groups, aryl groups, or heteroaryl groups, which themselves may be unsubstituted or substituted by one or more suitable substituents. Illustrative examples of heterocycloalkyl groups include, but are not limited to, azetidinyl, pyrrolidyl, piperidyl, piperazinyl, morpholinyl, tetrahydro-2H-1,4-thiazinyl, tetrahydrofuryl, dihydrofuryl, tetrahydropyranyl, dihydropyranyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, oxabicylo[2.2.1]heptyl, 1,5,9-triazacyclododecyl, and the like.

As used herein, “aryl” is intended to mean an aromatic, monovalent monocyclic, bicyclic, or tricyclic radical containing 6, 10, 14, or 18 carbon ring atoms, which may be unsubstituted or substituted by one or more suitable substituents as defined below or which may bear one or more C₁₋₆alkyl groups, and to which may be fused one or more cycloalkyl groups, heterocycloalkyl groups, or heteroaryl groups, which themselves may be unsubstituted or substituted by one or more suitable substituents. Illustrative examples of aryl groups include, but are not limited to, phenyl, tolulyl, naphthyl, fluoren-2-yl, indan-5-yl, and the like.

As used herein, “heteroaryl” is intended to mean an aromatic monovalent monocyclic, bicyclic, or tricyclic radical containing 5, 6, 7, 8, 9, 10, 11, 12, 13, 14, 15, 16, 17, or 18 ring atoms, including 1, 2, 3, 4, or 5 heteroatoms selected from nitrogen, oxygen, and sulfur, which may be unsubstituted or substituted by one or more suitable substituents as defined below or which may bear one or more C₁₋₆alkyl groups, and to which may be fused one or more cycloalkyl groups, heterocycloalkyl groups, or aryl groups, which themselves may be unsubstituted or substituted by one or more suitable substituents. Illustrative examples of heteroaryl groups include, but are not limited to, pyrrolyl, imidazolyl, pyrazolyl, furyl, thienyl, thiazolyl, oxazolyl, isoxazolyl, isothiazolyl, oxadiazolyl, triazolyl, tetrazolyl, pyrazinyl, pyridyl, pyrimidyl, pyridazinyl, indolyl, isoindolyl, benzimidazolyl, benzofuryl, isobenzofuryl, benzothienyl, quinolyl, isoquinolyl, phthalazinyl, carbazolyl, purinyl, pteridinyl, acridinyl, phenanthrolinyl, phenoxazinyl, phenothiazinyl, and the like.

As used herein, the term “suitable substituent” is intended to mean any of the radicals containing at least one atom other than just carbon and hydrogen that are recognizable to those skilled in the art as not adversely affecting the biological activity and/or stability of the inventive compounds. Illustrative examples of suitable substituents include, but are not limited to, oxo groups, acyl groups, alkoxy groups, aryloxy groups, carboxy groups, alkoxycarbonyl groups, carbamoyl groups, amino groups, alkylamino groups, dialkylamino groups, imine groups, hydroxy groups, halo groups, cyano groups, sulfonyl groups, sulfoxyl groups, nitro groups, heterocycloalkyl groups, heteroaryl groups, trialkylsilyl groups, thio groups, mercapto groups, and the like. The suitability of a given substituent may be determined empirically by one skilled in the art.

As used herein, “hydroxy” is intended to mean the radical —OH.

As used herein, “oxo” is intended to mean the divalent radical ═O.

As used herein, “acyl” is intended to mean a —C(O)R radical, wherein R is any alkyl as defined above.

As used herein, “halogen” or “halo” is intended to mean any of the radicals —F, —Cl, —Br, or —I.

As used herein, “cyano” is intended to mean the radical —C≡N.

As used herein, “sulfonyl” is intended to mean the radical —SO₂—R, wherein R is any alkyl as defined above.

As used herein, “sulfoxyl” is intended to mean the radical —S(O)R, wherein R is any alkyl as defined above.

As used herein, “nitro” is intended to mean the radical —NO₂.

As used herein, “trialkylsilyl” is intended to mean the radical —SiR₃, where each R is independently an alkyl.

As used herein, “carboxy” is intended to mean the radical —C(O)OH.

As used herein, “alkoxycarbonyl” is intended to mean the radical —C(O)OR, wherein R is an alkyl group as defined above.

As used herein, “carbamoyl” is intended to mean the radical —C(O)NH₂.

As used herein, “amino” is intended to mean the radical —NH₂.

As used herein, “alkylamino” is intended to mean the radical —NHR, where R is an alkyl group as defined above.

As used herein, “dialkylamino” is intended to mean the radical —NR₂, wherein each R is independently an alkyl group as defined above.

In view of the above, illustrative examples of substituted groups (i.e., groups substituted by one or more suitable substituents) in the context of the present invention include, but are limited to, the following. An alkyl group, such as a methyl group, may be substituted with three halogens, such as fluorine, to give, e.g., —CF₃. A methyl group may be substituted by an oxo group and an alkoxy group to give —C(O)-alkoxy, such as —C(O)—OC₁₋₆alkyl, or with an imine group and an amino group to give —C(NH)NH₂. A methyl group may be substituted with an oxo group and a hydroxyl group to give —C(O)OH. A methyl group may be substituted with an oxo group and an amino group to give —C(O)NH₂. A methyl group may be substituted with an oxo group and an alkylamino group to give —C(O)NHR, where R is an alkyl, to yield, e.g., —C(O)NH(C₁₋₆alkyl). A methyl group may be substituted with an oxo group and a dialkylamino group to give —C(O)NR₂, where R is an alkyl, to yield, e.g., —C(O)N(C₁₋₆alkyl)(C₁₋₆alkyl). The methyl group of a sulfonyl methyl group (i.e., —O—SO₂—CH₃) may be substituted with halogens to yield, e.g., —O—SO₂—CF₃.

Examples of hydroxy-substituted C₁₋₆alkyl groups include, but are not limited to, —CH₂CH(OH)CH₂CH₃ and —CH₂CH(OH)CH₂—O—CH(CH₃)₂. Examples of oxo- or carbamoyl-substituted C₁₋₆alkyl groups include, but are not limited to, —C(O)NHR′, where R′ may be, e.g., a halo-substituted benzyl group. Examples of thiocarbamoyl-substituted C₁₋₆alkyl groups include, but are not limited to, —C(S)NHR″, where R″ may be, e.g., a benzyl group or a halo-, alkoxy-, or alkyl-substituted benzyl group.

In the case of solid forms, it is understood that the inventive compounds may exist in one or more different forms, such as stable and metastable crystalline forms and isotropic and amorphous forms, individually or in a mixture, all of which are intended to be within the scope of the present invention. It is known in the art to isolate chemical compounds in any of such forms by slightly varying the method of purification and/or isolation from the solvents used in the synthetic preparation of such compounds.

As used herein, reference to a particular element shall also include all known isotopes thereof, unless the context clearly dictates otherwise. Thus, in the context of the present invention, reference to hydrogen should be understood to also refer to deuterium and tritium. Similarly, reference to carbon should be understood to refer to ¹³C and ¹⁴C in addition to ¹²C.

As used herein, the term “pharmaceutically acceptable prodrug” is intended to mean a compound that may be converted under physiological conditions or by solvolysis to a compound of formula (I). Examples of such prodrugs include compounds of formula (I) wherein one or more groups have been modified in a manner that is reversed upon administration to a human or other mammalian subject. Such reversion may be performed by an enzyme naturally present in such subject or simply by exposure to physiological conditions, such as the acidic environment of the stomach. Alternatively, a second agent may be administered to the subject together with a prodrug in order to perform the reversion in vivo. Examples of such modifications include esters, wherein the reversion may be carried out by an esterase, etc. Other such modifications are known to those skilled in the art.

As used herein, the term “solvate” is intended to include, but not be limited to, pharmaceutically acceptable solvates.

As used herein, the term “pharmaceutically acceptable solvate” is intended to mean a solvate that retains one or more of the biological activities and/or properties of the biologically active compounds of formula (I) and that is not biologically or otherwise undesirable. Examples of pharmaceutically acceptable solvates include, but are not limited to, compounds of formula (I) in combination with water, isopropanol, ethanol, methanol, DMSO, ethyl acetate, acetic acid, ethanolamine, or combinations thereof.

As used herein, the term “salt” is intended to include, but not be limited to, pharmaceutically acceptable salts

As used herein, the term “pharmaceutically acceptable salt” is intended to mean those salts that retain one or more of the biological activities and properties of the free acids and bases and that are not biologically or otherwise undesirable. Illustrative examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates.

If the inventive compound is a base, the desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acids such as glucuronic acid and galacturonic acid, alpha-hydroxy acids such as citric acid and tartaric acid, amino acids such as aspartic acid and glutamic acid, aromatic acids such as benzoic acid and cinnamic acid, sulfonic acids such as p-toluenesulfonic acid and ethanesulfonic acid, or the like.

If the inventive compound is an acid, the desired salt may be prepared by any suitable method known in the art, including treatment of the free acid with an inorganic or organic base, such as an amine (primary, secondary or tertiary), an alkali metal or alkaline earth metal hydroxide, or the like. Illustrative examples of suitable salts include organic salts derived from amino acids such as glycine and arginine, ammonia, primary, secondary and tertiary amines, and cyclic amines such as piperidine, morpholine and piperazine, and inorganic salts derived from sodium, calcium, potassium, magnesium, manganese, iron, copper, zinc, aluminum and lithium.

The inventive compounds may exist as single stereoisomers, racemates, and/or mixtures of enantiomers, and/or diastereomers. All such single stereoisomers, racemates, and mixtures thereof are intended to be within the scope of the present invention. These various forms of the compounds may be isolated/prepared by methods known in the art.

Preferably, the compounds of the present invention are prepared in a racemic mixture (i.e., mixture of isomers) that contains more than 50%, preferably at least 75%, and more preferably at least 90% of the desired isomer (i.e., 80% enantiomeric or diastereomeric excess). According to particularly preferred embodiments, the compounds of the present invention are prepared in a form that contains at least 95% (90% e.e. or d.e.), even more preferably at least 97.5% (95% e.e. or d.e.), and most preferably at least 99% (98% e.e. or d.e.) of the desired isomer. Compounds identified herein as single stereoisomers are meant to describe compounds used in a form that contains more than 50% of a single isomer. By using known techniques, these compounds may be isolated in any of such forms by slightly varying the method of purification and/or isolation from the solvents used in the synthetic preparation of such compounds.

As used herein, the term “pharmaceutically acceptable carrier” is intended to mean those carriers known and available to those skilled in the art that are suitable for the delivery of an active agent to a subject being treated and that are not biologically or otherwise undesirable.

The term “cell proliferation” disease or disorder is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, inflammatory disorders, fungal disorders, parasitic disorders such as malaria, emphysema, and alopecia.

The terms “treatment” or “treating”, as used herein, refer to anything that promotes or enhances the well-being of the subject with respect to their condition. A list of nonexhaustive examples of treatment benefits includes prophylaxis, prevention, and/or inhibition of a disorder or disease, extension of the subject's life by any period of time and/or decrease or delay in the development of the disease. Thus, for example, when the disease is cancer, “treatment of cancer” or “treating cancer” includes decreasing the growth and/or proliferation of cancer cells, reducing tumor growth, delaying and/or preventing metastases, reducing cancer cell proliferation, and decreasing pain to the subject that can be attributed to the subject's condition.

The term “reducing or inhibiting” is used variously herein to mean that a parameter is decreased due to an action of a compound of the invention as compared to the parameter in the absence of any action by a compound of the invention. For example, the term “reducing or inhibiting,” when used in reference to spindle fiber formation, means that the amount of spindle formation is decreased in cells treated with a compound of the invention as compared to untreated cells. Similarly, the term “reducing or inhibiting,” when used in reference to cell viability or to cell proliferation, means that survival or proliferative activity of cells contacted with a compound of the invention is less than the survival or proliferation in the absence of the compound. The terms “reduce” and “inhibit” are used together herein because it is recognized that, depending on the particular assay used to examine a parameter, the limit of detection of the assay may be such that it will not be able to be determined whether the parameter is inhibited or is reduced below the level of detection of the assay.

As used herein, the term “premitotic stage,” when used in reference to cells, means that the cells have not attained the anaphase stage of mitosis and, therefore, have not yet begun to divide.

### PARTICULARLY PREFERRED EMBODIMENTS

In a first particularly preferred embodiment, the present invention includes a compound of formula (I): 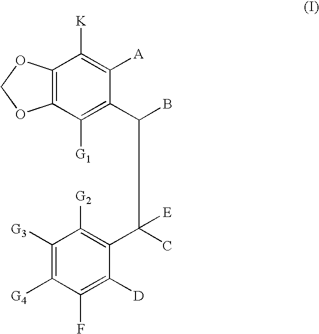
wherein: 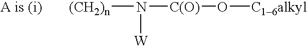
in which W is C₁₋₆alkyl or C₁₋₆alkylaryl and n=0, 1, or 2, or 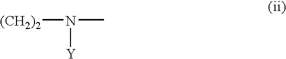
and forms a nitrogen-containing heterocycloalkyl ring with
B,
in which Y is:
   (a) hydrogen, C₁₋₆alkyl, or C₁₋₆alkylaryl,
   (b) —C(O)—C₁₋₆alkyl or —C(O)—C₁₋₆alkylaryl,
   (c) —CH₂—CH(OH)—CH₂-Z, where Z is C₁₋₆alkyl or —O—C₁₋₆alkyl,
   (d) aryl, or
   (e) heteroaryl;
B is —OH, halogen, or a single bond that forms a six-membered heterocycloalkyl ring with A;
C is hydrogen, C₁₋₆alkyl, or halogen;
D is (i) —CH₂-halogen, —CH(O), —COOH, —C(O)—O—C₁₋₆alkyl, —C(O)—O—C₁₋₆alkylaryl, —CH₂OH, or —(CH₂)ₙ—CH₃, wherein n is 1, 2, or 3, or
   (ii) together with E forms a five- or six-membered cycloalkyl or heterocycloalkyl ring;
E is —OH or C₁₋₆alkyl, or together with D forms a five- or six-membered cycloalkyl or heterocycloalkyl ring, wherein this heterocycloalkyl ring contains —C(O)O—, —C(O)NH—, —C(S)O—, or —C(S)NH—;
F is hydrogen, —O—C₁₋₆alkyl, —O—C₁₋₆alkylaryl, —O—C₁₋₆alkylheteroaryl, halogen, aryl, C₁₋₆alkyl, —SH, thio-C₁₋₆alkyl, —S-aryl, —O—SO₂—C₁₋₆alkyl, —O—SO₂—C₁₋₆alkylaryl, cyano, or NR1R2, where R1 and R2 are independently hydrogen, C₁₋₆alkyl, C₁₋₆alkylaryl, cyano, aryl, heteroaryl, —SO₂—C₁₋₆alkyl, or —SO₂—N(C₁₋₆alkyl)(C₁₋₆alkyl);
G₁ to G₄ independently represent hydrogen, aryl, halogen, C₁₋₆alkyl, hydroxyl, —S—C₁₋₆alkyl, nitro, —O—C₁₋₆alkyl, —O—C₁₋₆alkylaryl, or —(CH₂)ₓNR1R2, where x is 0, 1, or 2 and where R1 and R2 are independently hydrogen, C₁₋₆alkyl, C₁₋₆alkylaryl, cyano, aryl, heteroaryl, or acyl, or
two adjacent G₂ to G₄ groups together comprise an alkylene —(CH₂)ₘ—, where m is 3 or 4, to form a cycloalkyl ring, or together comprise an alkylene dioxy —O—(CH₂)ₙ—O—, where n is 1, 2, or 3, to form a heterocycloalkyl ring; and
K is C₁₋₆alkyl, halogen, cyano, aryl, hydrogen, hydroxyl, thio-C₁₋₆alkyl, sulfonyl, sulfoxyl, nitro, —O—C₁₋₆alkyl, —O—C₁₋₆alkylaryl, or NR1R2, where R1 and R2 are independently hydrogen, C₁₋₆alkyl, C₁₋₆alkylaryl, cyano, aryl, heteroaryl, or acyl;
wherein one or more of said alkyl, aryl, heteroaryl, cycloalkyl, heterocycloalkyl, and alkylaryl groups are optionally substituted with one or more suitable substituents;
   a salt thereof, a solvate thereof, a solvated salt thereof, or a combination of two or more thereof;
provided that when A is —(CH₂)₂—N(Y)— and forms a nitrogen-containing heterocycloalkyl ring with B, and D together with E forms an unsubstituted five-membered heterocycloalkyl ring that contains —C(O)O—, then:
   (i) F is not unsubstituted —O—C₁₋₆alkyl or dialkylamino-substituted —O—C₁₋₆alkyl when G₁ is hydrogen, hydroxyl, or unsubstituted —O—C₁₋₆alkyl, G₂ is hydrogen, halogen, or a nitrogen-containing radical, G₃ is hydrogen, G₄ is hydroxyl or unsubstituted —O—C₁₋₆alkyl, and Y is hydrogen, unsubstituted C₁₋₆alkyl, oxo-substituted C₁₋₆alkyl, thiocarbamoyl-substituted C₁₋₆alkyl, hydroxy-substituted C₁₋₆alkyl, or heteroaryl,
   (ii) F is not —NO₂ or NR1R2 where R1 and R2 are both hydrogen or the same oxo-substituted C₁₋₆alkyl (a) when at least three of G₁, G₂, G₃, and G₄ are the same unsubstituted —O—C₁₋₆alkyl or (b) when G₂ is —NO₂, and
   (iii) F is not hydrogen (a) when G₂, G₃, and G₄ are all hydrogen or (b) when G₂ and G₃ or G₃ and G₄ together comprise a methylenedioxy or (c) when at least two of G₂, G₃, and G₄ are unsubstituted —O—C₁₋₆alkyl or (d) when G₁ is unsubstituted —O—C₁₋₆alkyl and G₄ is a nitrogen-containing radical or halogen.

When any variable (e.g., W, Y, A, B, C, etc.) occurs more than one time in any constituents of the formulae of the present invention, its definition on each occurrence is independent of its definition at every other occurrence, unless otherwise specified. Also, combinations of substituents and/or other such variables are permissible only if such combinations result in stable compounds.

A preferred group of compounds of the present invention includes, but is not limited to, compounds of formula (I) in which A is —(CH₂)₂—N(Y)— and forms a nitrogen-containing heterocycloalkyl ring with B. The carbon atoms of such a heterocycloalkyl ring may each independently be unsubstituted or substituted with one or more suitable substitutents. More preferably, the carbon atoms of such a heterocycloalkyl ring are unsubstituted, and Y is hydrogen, C₁₋₆alkyl, or C₁₋₆alkylaryl, preferably C₁₋₆alkyl.

Yet another preferred group of compounds of the present invention includes, but is not limited to, compounds of formula (I) in which D, together with E, forms a substituted or unsubstituted five- or six-membered heterocycloalkyl ring that contains at least one of the following groups: —C(O)O—; —C(O)NH—; —C(S)O—; and —C(S)NH—. More preferably, D, together with E, forms a five-membered heterocycloalkyl ring that contains —C(O)O—, i.e., a lactone ring.

Still another preferred group of compounds of the present invention includes, but is not limited to, compounds of formula (I) in which A is —(CH₂)₂—N(Y)— and forms a nitrogen-containing heterocycloalkyl ring with B, and D, together with E, forms a substituted or unsubstituted five- or six-membered heterocycloalkyl ring that contains —C(O)O—, —C(O)NH—, —C(S)O—, or —C(S)NH—. More preferably, D, together with E, forms a five-membered heterocycloalkyl ring that contains —C(O)O—, i.e., a lactone ring.

Still other particularly preferred groups of compounds of the present invention include, but are not limited to, any of the above compounds or groups of compounds in which Y is hydrogen, C₁₋₆alkyl, or C₁₋₆alkylaryl.

Yet still other particularly preferred groups of compounds of the present invention include, but are not limited to, any of the above compounds or groups of compounds in which K is hydrogen.

Yet still other particularly preferred groups of compounds of the present invention include, but are not limited to, any of the above compounds or groups of compounds in which one or more of G₁ to G₄ each independently represents hydrogen or —O—C₁₋₆alkyl.

According to certain preferred embodiments, G₁ and G₄ are the same. According to such embodiments, G₁ and G₄ are preferably each —O—C₁₋₆alkyl, and more preferably contain the same alkyl moiety, e.g., methyl, ethyl, n-propyl, iso-propyl, and the like.

According to other preferred embodiments, G₂ and G₃ are the same. According to such embodiments, G₂ and G₃ are preferably each hydrogen.

The compounds of the present invention may be made by any of the various methods and techniques known and available to those skilled in the art. Particularly preferred methods will now be described in greater detail.

Direct Nucleophilic Substitution

According to certain preferred embodiments, the compounds of the present invention may be formed by direct nucleophilic substitution of the appropriate precursor compound. A suitable direct nucleophilic substitution process may comprise reacting a compound of formula (IV): 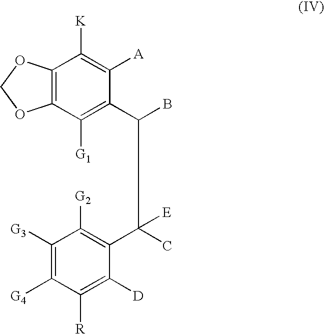
wherein each of the variables other than R may be as defined above and R is a suitable leaving group, for example halogen, —O—C₁₋₆alkyl, and —O—SO₂—C₁₋₆alkyl (including substituted —O—SO₂—C₁₋₆alkyl such as —O—SO₂—CF₃) with a suitable nucleophile to form a compound of formula (V): 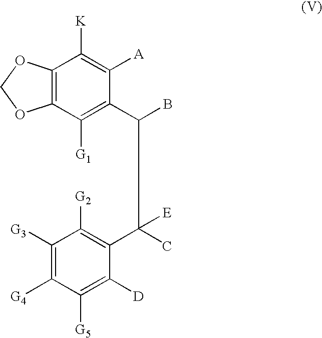
wherein G₅ is the radical derived from the suitable nucleophile employed and may be, for example, aryl, halogen, C₁₋₆alkyl, hydroxyl, C₁₋₆alkylaryl, thio-C₁₋₆alkyl, nitro, —O—C₁₋₆alkyl, —O—C₁₋₆alkylaryl, —SH, —S-aryl, or —(CH₂)ₓNR1R2, where x is 0, 1, or 2 and R1 and R2 are each independently hydrogen, C₁₋₆alkyl, C₁₋₆alkylaryl, cyano, aryl, heteroaryl, or carboxy.

In a preferred embodiment of direct nucleophilic substitution, trifluoromethanesulfonic acid 5-methoxy-1-(4-methoxy-5,6,7,8-tetrahydro-[1,3]dioxolo[4,5-g]isoquinolin-5-yl)-3-oxo-1,3-dihydro-isobenzofuran-4-yl ester (7-OTf noscapine) is employed as the starting compound of formula (IV) for direct nucleophilic substitution reactions. The 7-OTf noscapine may be converted to other compounds by known methods.

For example, 7-OTf noscapine may optionally be combined with a suitable catalyst. Examples of catalysts include tris(dibenzylideneacetone)-dipalladium chloroform adduct, 1,1′-bis(diphenylphosphino)ferrocene (DPPF), tetrakis(triphenylphosphine)palladium, and mixtures thereof. The 7-OTf noscapine and catalyst may be combined under suitable conditions, such as by heating to a temperature of at least 90° C.

A suitable base, such as triethylamine or (2′-dicyclohexylphosphanyl-biphenyl-2-yl)-dimethyl-amine, may then be added to the mixture, e.g., in a molar ratio of 7-OTf noscapine to base of about 1:1.1 (i.e., slight excess of base), under suitable conditions.

Other reaction conditions, such as the appropriate temperature and pressure employed as well as the time period over which the reaction is allowed to proceed, may be determined and optimized empirically by one skilled in the art. For example, when 7-OTf noscapine is employed as the starting material, the reaction may be allowed to proceed for a period of about 15 hours, optionally while being heated to a temperature of about 80° C. to 90° C. at ambient pressure.

In another direct nucleophilic substitution process, compounds of formula (IV) may be treated with a variety of alkali or alkaline earth alkoxides, such as sodium alkoxide, to form a reaction mixture. In particularly preferred embodiments, noscapine is employed as the starting compound of formula (IV) for direct nucleophilic substitution reactions.

The reaction mixture is then preferably allowed to react under suitable conditions for a period of time sufficient to effect the desired transformation. One skilled in the art may determine appropriate reaction conditions empirically, based, at least in part, on the particular diastereomer, or ratio of diastereomers, desired, as well as the particular reactants employed.

For example, when noscapine (a commercially available material) is employed as the starting material, exemplary products of direct nucleophilic substitution include diastereomers 2a and/or 2b:

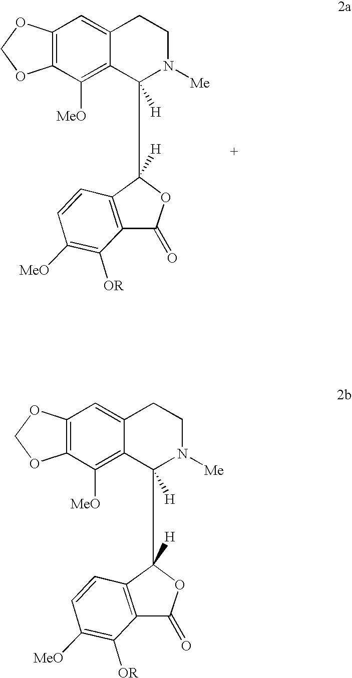

According to certain embodiments of the present invention, an anhydrous alcohol/alkoxide system is employed in the above reaction (such as anhydrous methanol/sodium methoxide) and diastereomer 2a is preferentially formed. According to other embodiments, an aqueous alkaline system is employed (such as methanol/potassium hydroxide) and diastereomer 2b is preferentially formed.

Other reaction conditions, such as the appropriate temperature and pressure employed as well as the time period over which the reaction is allowed to proceed, may be determined and optimized empirically by one skilled in the art. For example, when noscapine is employed as the starting material, the reaction may be allowed to proceed for a period ranging from about 15 to 20 minutes, optionally while being heated to a temperature ranging from about 120° C. to 130° C. at ambient pressure.

Direct Alkylation

According to certain preferred embodiments, the compounds of the present invention may be formed by direct alkylation of the appropriate precursor compound. A suitable direct nucleophilic substitution process may comprise reacting a compound of formula (VI): 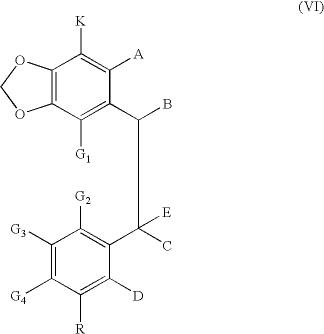
wherein each of the variables other than R may be as defined above and R is a suitable leaving group, for example hydroxyl or substituted hydroxyl, with a suitable donor to form a compound of formula (VII): 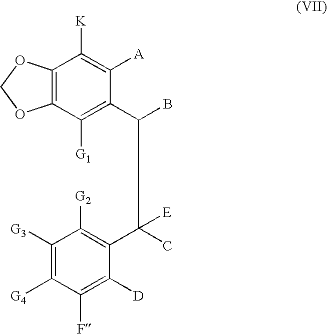
wherein F″ is the radical derived from the suitable donor employed and may be, for example, —O—C₁₋₆alkyl or substituted —O—C₁₋₆alkyl, such as —O—C₁₋₆alkylaryl or —O—C₁₋₆alkylheteroaryl.

In a preferred embodiment of direct alkylation, 7-hydroxy noscapine is employed as the starting compound of formula (VI) for direct nucleophilic substitution reactions. The 7-hydroxy noscapine may be converted to other compounds by known methods.

For example, the 7-hydroxy noscapine may be combined with at least one suitable donor and preferably also with at least one suitable catalyst or base. Examples of suitable catalysts include tetrabutylammonium iodide. Examples of suitable donors include alkyl halides, such as 4-(chloromethyl)-2-methyl-1,3-thiazole and 3,4,5-trimethoxybenzyl chloride.

Reaction conditions, such as the appropriate temperature and pressure employed as well as the time period over which the reaction is allowed to proceed, may be determined and optimized empirically by one skilled in the art. For example, when 7-hydroxy noscapine is employed as the starting material, the reaction may be allowed to proceed for a period of about 18 hours, optionally while being heated to a temperature of about 80° C. to 90° C. at ambient pressure.

### Synthesis through regio- and stereoselective O-dealkylation:

Compounds of the present invention that contain hydroxyl groups, which are useful per se or as intermediates to form other compounds of the present invention, may be formed from alkyl ether-containing precursors. Such conversion may be performed using any suitable reagent effective in selectively cleaving the desired alkyl ether bond without adversely affecting any other part of the molecule. For instance, the reagent may be an alkyl Grignard reagent.

Such O-dealkylation preferably comprises converting a compound of formula (VIII): 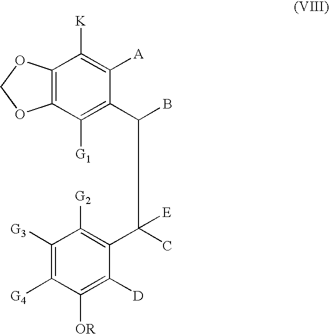
wherein variables other than R may be as defined above, and R is a C₁₋₆alkyl group;
into a compound of formula (IX):

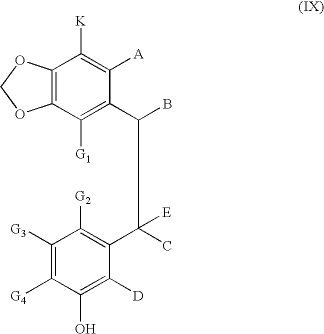

Thus, for example, when the compound of formula (VIII) is noscapine or a derivative thereof, it may be combined with an alkyl Grignard reagent, such as methyl magnesium bromide, preferably in a high boiling alcohol (e.g., having a boiling point over 140° C.), such as benzyl alcohol, under anhydrous conditions to form a reaction mixture. According to such an embodiment, the alcohol, Grignard reagent, and the noscapine or derivative thereof are preferably combined in a molar ratio of 3:1.2:1.

Such a reaction mixture may also include at least one suitable solvent, such as N-methylpyrrolidinone, dimethylformamide, tetrahydrofuran, and/or toluene.

Such a reaction mixture may then be permitted to react at a suitable temperature, such as 120° C., for a suitable period, such as 16 hours.

The above selective dealkylation may be the first step used to convert a compound of formula (II): 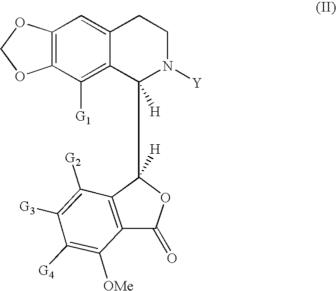
wherein:
Y is:
   (a) hydrogen, C₁₋₆alkyl, or C₁₋₆alkylaryl,
   (b) —C(O)—C₁₋₆alkyl or —C(O)—C₁₋₆alkylaryl,
   (c) —CH₂—CH(OH)—CH₂-Z, where Z is C₁₋₆alkyl or —O—C₁₋₆alkyl,
   (d) aryl, or
   (e) heteroaryl; and
G₁ to G₄ independently represent hydrogen, aryl, halogen, C₁₋₆alkyl, hydroxyl, —S—C₁₋₆alkyl, nitro, —O—C₁₋₆alkyl, —O—C₁₋₆alkylaryl, or —(CH₂)ₓNR1R2, where x is 0, 1, or 2 and where R1 and R2 are independently hydrogen, C₁₋₆alkyl, C₁₋₆alkylaryl, cyano, aryl, heteroaryl, or acyl, or
two adjacent G₂ to G₄ groups together comprise an alkylene —(CH₂)ₘ—, where m is 3 or 4, to form a cycloalkyl ring, or together comprise an alkylene dioxy —O—(CH₂)ₙ—O—, where n is 1, 2, or 3, to form a heterocycloalkyl ring;
a salt thereof, a solvate thereof, a solvated salt thereof, or a combination of two or more thereof;
into a single stereoisomer of formula (III): 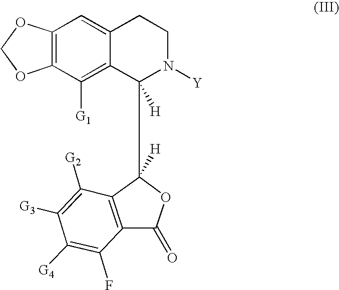
   wherein G₁, G₂, G₃, G₄, and Y are as defined above, and F is —O—C₂₋₆alkyl, —O—C₁₋₆alkylaryl, —O—C₁₋₆alkylheteroaryl, halogen, aryl, C₁₋₆alkyl, —SH, thio-C₁₋₆alkyl, —S-aryl, —O—SO₂—C₁₋₆alkyl, —O—SO₂—C₁₋₆alkylaryl, cyano, or NR1R2, where R1 and R2 are independently hydrogen, C₁₋₆alkyl, C₁₋₆alkylaryl, cyano, aryl, heteroaryl, —SO₂—C₁₋₆alkyl, or —SO₂—N(C₁₋₆alkyl)(C₁₋₆alkyl), provided that F is not —O-t-C₄H₉ or —O—CH₂CH₂N(C₂H₅)₂;
wherein one or more of said alkyl, aryl, heteroaryl, and alkylaryl groups are optionally substituted with one or more suitable substituents;
a salt thereof, a solvate thereof, a solvated salt thereof, or a combination of two or more thereof.

According to certain preferred embodiments, the yield of the converting is at least 60%, preferably at least 70%, more preferably at least 80%, even more preferably at least 90%, still even more preferably at least 95%, and most preferably at least 99%.

According to still other preferred embodiments, the desired stereoisomer is obtained in an enantiomeric or diastereomeric excess, and preferably an excess of at least 9:1 relative to any other stereoisomer(s) produced.

For instance, after the first step of selective dealkylation, the resulting hydroxyl compound may be reacted as known in the art. Furthermore, the resulting hydroxyl compound may be reacted as described herein. For example, a 7-hydroxy compound, such as 7-hydroxy noscapine, may be directly alkylated to give the corresponding O-alkyl derivatives without affecting the stereochemistry of any chiral centers.

Alkoxide Addition

According to other preferred embodiments, the compounds of the present invention may also be formed by alkoxide addition. A suitable alkoxide addition process may comprise converting a compound of formula (X): 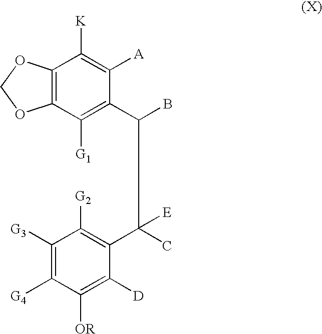
wherein variables other than R may be as defined above and R is a C₁₋₆alkyl group, into a compound of formula (XI): 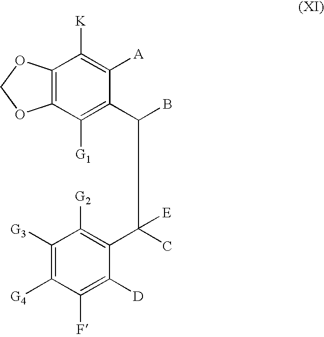
wherein F′ is —O—C₁₋₆alkyl (in which the alkyl moiety differs from R), —O—C₁₋₆alkylaryl, or —O—C₁₋₆alkylheteroaryl, such as thiophene or thiazole.

According to certain preferred embodiments of such a process, a compound of formula (X) may be reacted with a base in a suitable solvent (e.g., toluene and/or 1-methyl-2-pyrrolidinone), to form an alkoxide. The alkoxide may then be reacted with an appropriate electrophilic alkylating agent, such as an alkyl halide, preferably in the presence of a base (e.g., a metal hydride such as NaH), to form a compound of formula (XI).

According to certain preferred embodiments, the compound of formula (X) and electrophilic alkylating agent are combined in a molar ratio ranging from about 1:1 to about 1:10, such as about 1:1.5. When a base is present in the reaction, it is combined with the compound of formula (X) in a molar ratio ranging from about 1:1 to about 1:3, such as about 1:1.2.

A preferred illustrative example of a suitable compound of formula (X) for use in aromatic alkylation processes is, but is not limited to, noscapine.

Illustrative examples of suitable electrophilic alkylating agents include, but are not limited to, alkylaryl halides, such as 3,4,5-trimethoxybenzyl chloride, and heteroaryls, such as thiophene and thiazole.

One skilled in the art may determine suitable reaction conditions empirically based, in part, on the particular compound of formula (X) and/or particular electrophilic alkylating agent employed. For example, the reaction may be allowed to proceed for a period ranging from about 12 to 18 hours, optionally while being heated to a temperature ranging from about 80° C. to 90° C. at ambient pressure.

Preferred products of such alkoxide addition processes include, but are not limited to, the following: 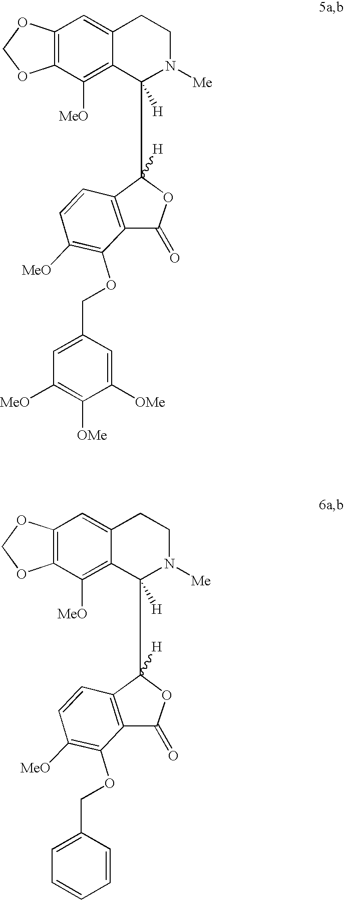
N-alkylation

Compounds in which the nitrogen atom bears a hydrogen may be alkylated as follows. A secondary amine-containing compound may be reacted with an electrophilic alkylating agent, optionally in the presence of a base.

According to certain preferred embodiments, the secondary amine-containing compound and electrophilic alkylating agent are combined in a molar ratio ranging from about 1:1.5 to about 1:4, such as about 1:3. When a base is present in the reaction, it may be combined with the secondary amine-containing compound in a molar ratio ranging from about 1:10 to about 1:20, such about 1:15.

Preferred illustrative examples of suitable secondary amine-containing compounds for use in N-alkylation processes include, but are not limited to, N-demethylated noscapine (i.e., 6,7-dimethoxy-3-(4-methoxy-5,6,7,8-tetrahydro-[1,3]dioxolo[4,5-g]isoquinolin-5-yl)-3H-isobenzofuran-1-one) and N-demethylated 7-hydroxy noscapine (i.e., 7-hydroxy-6-methoxy-3-(4-methoxy-5,6,7,8-tetrahydro-[1,3]dioxolo[4,5-g]isoquinolin-5-yl)-3H-isobenzofuran-1-one).

Illustrative examples of suitable electrophilic alkylating agents include, but are not limited to, benzyl halides, such as 3,4,5-trimethoxybenzyl chloride, and heteroaryls, such as thiophene and thiazole.

One skilled in the art may determine suitable reaction conditions empirically based, in part, on the particular N-demethylated compound and/or particular electrophilic alkylating agent employed. For example, the reaction may be allowed to proceed for a period ranging from about 10 to 18 hours, optionally while being heated to a temperature ranging from about 50° C. to 60° C. at ambient pressure.

An exemplary product of such N-alkylation processes is, but is not limited to, the following: 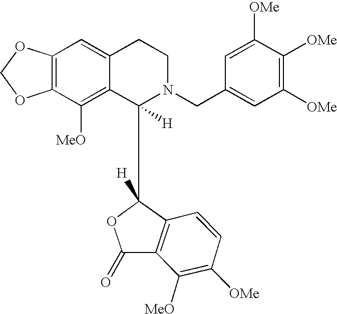
Amine and Amino Derivatives:

According to other preferred embodiments, amino derivatives may be formed from the corresponding hydroxy compound by any of the methods known to those skilled in the art. See, e.g., WOLFE et al., J.Org.Chem., 65:1158-1174 (2000) and WOLFE et al., J.Org.Chem., 62:1264-1267 (1997). Such amines may then optionally likewise be converted to the corresponding amino compound by any of the methods known to those skilled in the art.

For example, a suitable hydroxy compound (e.g., 7-hydroxy noscapine) may be converted into the corresponding triflate derivative by known methods, such as by combining the hydroxy compound with triflic anhydride in the presence of a suitable base. This triflate derivative may then subsequently be converted to the desired amine (e.g., 7-amino noscapine) by known methods. Secondary amines or tertiary amines produced by such a process may optionally be reduced to the corresponding primary amine by known methods, such as reduction with hydrogen gas in the presence of palladium on activated carbon.

Cyano Compounds:

The triflate derivatives of the present invention, such as 7-OTf noscapine, may also be used to form cyano compounds. Such a conversion may be achieved by known methods, such as by combining a triflate derivatve with a suitable catalyst, such as a mixture of tris(dibenzylideneacetone)-dipalladium chloroform adduct and 1,1′-bis(diphenylphosphino)ferrocene (DPPF), under suitable conditions, such as by heating to a temperature of at least 90° C. A suitable cyanide donor, such as zinc cyanide, may then be added to the mixture, e.g., in a molar ratio of 1:1.1, under suitable conditions.

Aryl Compounds:

The triflate derivatives of the present invention may also be used to form aryl compounds. Such compounds may be prepared by methods known to those skilled in the art. For example, a suitable triflate derivative, such as 7-triflate noscapine, may be combined with a suitable substituted aryl reactant, such as 3,5-difluorophenylboronic acid, preferably in the presence of at least one catalyst, such as a mixture of tetrakis(triphenylphosphine) palladium and lithium chloride, and at least one base.

Triflate Reduction

The triflate derivatives of the present invention may also be reduced. Such compounds may be prepared by methods known to those skilled in the art. For example, a suitable triflate derivative, such as 7-triflate noscapine, may be combined with a suitable base, such as triethylamine, preferably in the presence of at least one catalyst, such as a mixture of palladium acetate and 1,3-bis(diphenylphosphino)propane.

Hydrosulfide Addition

The triflate derivatives of the present invention may also be used to form mercapto compounds. Such compounds may be prepared by methods known to those skilled in the art. For example, a suitable triflate derivative, such as 7-triflate noscapine, may be combined with a suitable reactant, such as sodium hydrosulfide.

Purification

The intermediates and products of the above reaction schemes may be purified and/or separated as desired. One skilled in the art may select from any of the known and available purification and/or separation techniques, alone or in combination(s).

For instance, the compounds of the present invention may be separated and/or purified from reaction mixtures by diluting with a suitable organic solvent, such as ethyl acetate, and then washing the diluted organic solution with water or aqueous salt solutions to remove water-soluble impurities and reactants. The remaining organic layer may be dried by known methods (e.g., by adding magnesium sulfate) and then preferably concentrated (e.g., by application of reduced pressure).

Another suitable separation and/or purification technique involves adding water or an aqueous acid solution, such as hydrochloric acid, acetic acid, citric acid, sulfuric acid, or the like, to the reaction mixture containing one or more compounds of the present invention. If an acid is used, the aqueous layer is preferably rendered alkaline (i.e., a pH of at least 8) by adding an effective amount of a suitable base, such as sodium carbonate. The resulting mixture may then be extracted with a suitable organic solvent, such as diethyl ether, dichloromethane, and/or ethyl acetate, and the recovered organic fractions dried (e.g., over sodium sulfate), decanted or filtered, and then concentrated (e.g., by vacuum).

When a catalyst is used in the reaction, the following technique may be appropriate. The catalyst may be filtered off, and the filtrate may then be concentrated, e.g., by vacuum.

Other suitable methods for purifying and/or separating the compounds of the present invention include, but are not limited to, silica gel chromatography, flash chromatography, liquid chromatography mass spectrometry (LCMS), and/or RP-HPLC methods.

Methods of Use

The present invention is also directed to methods of using the compounds of the present invention for treatment of diseases and disorders associated with cell cycle progression, cell proliferation, tissue hyperplasia, angiogenesis, or a combination thereof, such as cancer, papillomas, acute and chronic inflammation, rheumatoid arthritis, psoriasis, atherosclerosis, diabetic retinopathy, chronic obstructive pulmonary disorder, tuberculosis, chronic cholecystitis, osteoarthritis, rheumatic carditis, bronchiectasis, Hashimoto's thyroiditis, inflammatory bowel diseases such as ulcerative colitis and Crohn's disease, silicosis, and the like.

In cell proliferation disorders, the compounds of the present invention may induce apoptosis or maintain stasis within the desired cells as required.

Accordingly, in one aspect, the present invention includes a method of treating cancer by administering to a mammal in need thereof an effective amount of a compound of formula (I), a pharmaceutically acceptable salt thereof, a pharmaceutically acceptable solvate thereof, a pharmaceutically acceptable prodrug thereof, a pharmaceutically acceptable solvated salt thereof, a pharmaceutically acceptable solvated prodrug thereof, a pharmaceutically acceptable salt of a prodrug thereof, or a combination of two or more thereof.

While not wishing to be bound by any theory of operability, compounds of formula (I) arrest dividing cells at the S-phase or G2/M-phase. Since the cell cycle or cell division is arrested, the cells undergo apoptosis.

Preferred compounds of the present invention which arrest dividing cells at the S-phase or G2-M phase include compounds of formula (I) wherein A is —(CH₂)₂—N(Y)— and forms a nitrogen-containing heterocycloalkyl ring with B.

Still other preferred compounds of the present invention which arrest dividing cells at the S-phase or G2-M phase include compounds of formula (I) wherein D together with E forms a substituted or unsubstituted five- or six-membered heterocycloalkyl ring that contains —C(O)O—, —C(O)NH—, —C(S)O—, or —C(S)NH—.

Still other preferred compounds of the present invention which arrest dividing cells at the S-phase or G2-M phase include compounds of formula (I) wherein D together with E forms a five-membered heterocycloalkyl ring that contains —C(O)O—.

Particularly preferred compounds of the present invention which arrest dividing cells at the S-phase or G2-M phase include compounds of formula (I) wherein A is —(CH₂)₂—N(Y)— and forms a nitrogen-containing heterocycloalkyl ring with B, and D together with E forms a substituted or unsubstituted five- or six-membered heterocycloalkyl ring that contains —C(O)O—C(O)NH—, —C(S)O—, or —C(S)NH—.

Even more particularly preferred compounds of the present invention which arrest dividing cells at the S-phase or G2-M phase include compounds of formula (I) wherein A is —(CH₂)₂—N(Y)— and forms a nitrogen-containing heterocycloalkyl ring with B, and D together with E forms a five-membered heterocycloalkyl ring that contains —C(O)O—.

Preferred compounds of the present invention which cause cells to accumulate in S-phase include compounds of formula (I) wherein F is O—C₁₋₆alkylaryl, which may be unsubstitued, e.g., benzyloxy, or substituted, e.g., 3,4,5-trimethoxybenzyloxy.

Preferred compounds of the present invention which cause cells to accumulate in G2/M-phase include compounds of formula (I) wherein F is hydrogen, —SH, NR1R2, or cyano.

Compounds of formula (I) reduce or inhibit tubulin polymerization. As a result, spindle fiber formation is reduced or inhibited and the cells are blocked in a premitotic stage. Thus, inhibition of spindle formation results in mitotic arrest.

Preferred compounds of the present invention which reduce or inhibit tubulin polymerization include compounds of formula (I) wherein A is —(CH₂)₂—N(Y)— and forms a nitrogen-containing heterocycloalkyl ring with B.

Still other preferred compounds of the present invention which reduce or inhibit tubulin polymerization include compounds of formula (I) wherein D together with E forms a substituted or unsubstituted five- or six-membered heterocycloalkyl ring that contains —C(O)O—, —C(O)NH—, —C(S)O—, or —C(S)NH—.

Still other preferred compounds of the present invention which reduce or inhibit tubulin polymerization include compounds of formula (I) wherein D together with E forms a five-membered heterocycloalkyl ring that contains —C(O)O—.

Particularly preferred compounds of the present invention which reduce or inhibit tubulin polymerization include compounds of formula (I) wherein A is —(CH₂)₂—N(Y)— and forms a nitrogen-containing heterocycloalkyl ring with B, and D together with E forms a substituted or unsubstituted five- or six-membered heterocycloalkyl ring that contains —C(O)O—, —C(O)NH—, —C(S)O—, or —C(S)NH—.

Even more particularly preferred compounds of the present invention which reduce or inhibit tubulin polymerization include compounds of formula (I) wherein A is —(CH₂)₂—N(Y)— and forms a nitrogen-containing heterocycloalkyl ring with B, and D together with E forms a five-membered heterocycloalkyl ring that contains —C(O)O—.

Still even more particularly preferred compounds of the present invention which reduce or inhibit tubulin polymerization include compounds of formula (I) wherein F is NR1R2.

Thus, the present invention also provides methods of reducing or inhibiting the ability of the cells to proceed through mitosis and, therefore, proliferate, by contacting the cells with a compound of the invention. In addition, since other tubulin ligands have general applications in anti-restenosis, anti-fungal, anti-helminths, and anti-gout chemotherapies, the present invention also provides for the treatment of such conditions.

For example, the ability of the cells to proceed through mitosis in the presence of a compound of the invention may be completely inhibited, i.e., 100% of the cells are blocked in a premitotic stage, or may be reduced such that 99%, 95%, or 90% of the cells are blocked in a premitotic stage. It should be recognized, however, that regardless of whether the recited parameter is “reduced” or is “inhibited,” the level of the parameter as determined in the presence of a compound of the invention will be measurably decreased as compared to the level the parameter would be in the absence of the compound.

Because compounds of the present invention cause cells to accumulate in a premitotic stage, the compounds may be used to prepare a population of cells for examining chromosome structure in the cells. Moreover, such cells are also suitable, for example, for chromosome staining using well known methods such as Giemsa staining or quinacrine staining to perform G band or C band analysis or the like. In addition, the ability to increase the percentage of cells in a premitotic stage allows for the isolation of a synchronized population of cells, for example, by fluorescence activated cell sorting or, where the cells normally attach to a tissue culture plate, by shaking rounded, detached premitotic cells from the plate, as is known in the art.

Compounds of formula (I) also affect topoisomerases. Without wishing to be bound by any theory of operability, topoisomerase inhibitors generally act by stabilization of the cleavage complex formed by the enzyme attached to the strand of DNA, which leads to the production of irreversible cleavages of the DNA and triggers cell apoptosis.

Topoisomerase I causes a single-strand break that can allow the two sections of DNA helix to rotate relative to each other. Targeting the nuclear enzyme topoisomerase I may cause single and double strand-DNA breaks and subsequent cell death.

The essential nuclear enzyme topoisomerase II allows the separation of intertwined DNA strands by creating a transient double stranded break in the DNA backbone. Without wishing to be bound by any theory of operability, compounds of the present invention may operate via stabilization of the topoisomerase II-DNA complex and/or interaction with G-quadraplexes. Moreover, the compounds of the present invention may inhibit the relegation step of topoisomerase II at a step where the enzyme has created a cleavable complex in DNA. Similarly, the antibiotic activity of the compounds of the present invention may derive from their ability to interact with the gyrase-DNA complex, which is the bacterial type II topoisomerase.

Preferred compounds of the present invention which inhibit topoisomerases, such as topoisomerase I and/or topoisomerase II, include compounds of formula (I) wherein A is —(CH₂)₂—N(Y)— and forms a nitrogen-containing heterocycloalkyl ring with B.

Still other preferred compounds of the present invention which inhibit topoisomerases, such as topoisomerase I and/or topoisomerase II, include compounds of formula (I) wherein D together with E forms a substituted or unsubstituted five- or six-membered heterocycloalkyl ring that contains —C(O)O—, —C(O)NH—, —C(S)O—, or —C(S)NH—.

Still other preferred compounds of the present invention which inhibit topoisomerases, such as topoisomerase I and/or topoisomerase II, include compounds of formula (I) wherein D together with E forms a five-membered heterocycloalkyl ring that contains —C(O)O—.

Particularly preferred compounds of the present invention which inhibit topoisomerases, such as topoisomerase I and/or topoisomerase II, include compounds of formula (I) wherein A is —(CH₂)₂—N(Y)— and forms a nitrogen-containing heterocycloalkyl ring with B, and D together with E forms a substituted or unsubstituted five- or six-membered heterocycloalkyl ring that contains —C(O)O—, —C(O)NH—, —C(S)O—, or —C(S)NH—.

Even more particularly preferred compounds of the present invention which inhibit topoisomerases, such as topoisomerase I and/or topoisomerase II, include compounds of formula (I) wherein A is —(CH₂)₂—N(Y)— and forms a nitrogen-containing heterocycloalkyl ring with B, and D together with E forms a five-membered heterocycloalkyl ring that contains —C(O)O—.

Still even more particularly preferred compounds of the present invention which inhibit topoisomerases, such as topoisomerase I and/or topoisomerase II, include compounds of formula (I) wherein F is O—C₁₋₆alkyl, such as methoxy, O—C₁₋₆alkylaryl, which is preferably substituted, such as 3,4,5-trimethyoxybenzyl, or O—C₁₋₆alkylheteroaryl, such as 2-thiophenyl or 2-methyl-1,3-thiazol-4-yl.

Still even more particularly preferred compounds of the present invention which inhibit topoisomerases, such as topoisomerase I and/or topoisomerase II, include compounds of formula (I) wherein Y is C₁₋₆alkyl, such as methyl, C₁₋₆alkylaryl, preferably substituted C₁₋₆alkylaryl such as 3,4,5-trimethoxybenzyl.

The compounds of formula (I) are therefore generally useful in the treatment of tumor cells and a variety of cancers, including, but not limited to, cancer of the colon, non-small cell lung cancer, cancer of the brain, ovarian cancer, cervical cancer, cancer of the kidney, cancer of the prostate, leukemia, breast cancer, skin cancer, melanoma, and cancer of the bladder.

In addition, the compounds of the present invention may also be useful as antitussives in treating a cough, particularly in mammals such as humans. These compounds may further be used for treatment of allergies, hemorrhages, pain, viruses, protozoas, high cholesterol, stuttering, and liver conditions.

A preferred group of compounds for use in the methods of the present invention includes compounds of formula (I) in which A is —(CH₂)₂—N(Y)— and forms a nitrogen-containing heterocycloalkyl ring with B. The carbon atoms of such a heterocycloalkyl ring may each independently be unsubstituted or substituted with one or more suitable substituents. More preferably, the carbon atoms of such a heterocycloalkyl ring are unsubstituted, and Y is unsubstituted or substituted C₁₋₆alkyl or C₁₋₆alkylaryl.

Yet another preferred group of compounds for use in the methods of the present invention includes compounds of formula (I) in which D, together with E, forms a substituted or unsubstituted five- or six-membered heterocycloalkyl ring that contains at least one of the following groups: —C(O)O—; —C(O)NH—; —C(S)O—; and —C(S)NH—. More preferably, D, together with E, forms a five-membered heterocycloalkyl ring that contains —C(O)O—, i.e., a lactone ring.

Still another preferred group of compounds for use in the methods of the present invention includes compounds of formula (I) in which A is —(CH₂)₂—N(Y)— and forms a nitrogen-containing heterocycloalkyl ring with B, and D, together with E, forms a substituted or unsubstituted five- or six-membered heterocycloalkyl ring that contains —C(O)O—, —C(O)NH—, —C(S)O—, or —C(S)NH—. More preferably, D, together with E, forms a five-membered heterocycloalkyl ring that contains —C(O)O—, i.e., a lactone ring.

Still other particularly preferred groups of compounds for use in the methods of the present invention include any of the above compounds or groups of compounds in which Y is hydrogen, C₁₋₆alkyl, or C₁₋₆alkylaryl.

Yet still other particularly preferred groups of compounds for use in the methods of the present invention include, but are not limited to, any of the above compounds or groups of compounds in which K is hydrogen.

Yet still other particularly preferred groups of compounds for use in the methods of the present invention include, but are not limited to, any of the above compounds or groups of compounds in which one or more of G₁ to G₄ each independently represents hydrogen or —O—C₁₋₆alkyl.

According to certain preferred embodiments, G₁ and G₄ are the same. According to such embodiments, G₁ and G₄ are preferably each —O—C₁₋₆alkyl, and more preferably contain the same alkyl moiety, e.g., methyl, ethyl, n-propyl, iso-propyl, and the like.

According to other preferred embodiments, G₂ and G₃ are the same. According to such embodiments, G₂ and G₃ are preferably each hydrogen.

Pharmaceutical Compositions

In the methods of the present invention, the compounds of the present invention can be delivered or administered to a mammal, e.g., a human patient, alone, or in the form of a pharmaceutical composition where the compound is mixed with suitable carriers or excipient(s).

The compounds of formula (I) can be incorporated into a variety of formulations for therapeutic administration. The compounds of formula (I) are generally delivered in dosage unit formulations containing non-toxic pharmaceutically acceptable carriers, adjuvants, and vehicles. For example, the compounds of formula (I) may be formulated into pharmaceutical compositions by combination with appropriate, pharmaceutically acceptable carriers or diluents, and may be formulated into preparations in solid, semi-solid, liquid or gaseous forms, such as orally-administerable suspensions, tablets, capsules, pills, powders, granules, dragees, gels, slurries, ointments, solutions, elixirs, suppositories, injections (e.g., sterile injectable aqueous), inhalants, and aerosols.

As such, administration of the compounds may be achieved in various ways, including oral, buccal, rectal, nasal (e.g., by inhalation spray), parenteral (including subcutaneous injections, intravenous, intramuscular, infrasternal injection or infusion techniques), intraperitoneal, intradermal, transdermal, intratracheal, topical delivery, etc., administration, and combinations thereof. Moreover, the compounds may also be administered in a local rather than systemic manner, for example via injection of the compound directly into a solid tumor, often in a depot or sustained release formulation. In addition, the compounds can be administered via a targeted drug delivery system, for example, in a liposome coated with tumor-specific antibody. Such liposomes are targeted to and taken up selectively by the tumor.

Suitable formulations for use in the present invention are found in Remington's Pharmaceutical Sciences, 17th ed. (1985). Moreover, for a brief review of methods for drug delivery, see LANGER, Science 249:1527-1533 (1990). The pharmaceutical compositions useful in the methods of the present invention may be manufactured in a known manner, e.g., by means of mixing, dissolving, granulating, dragee-making, levigating, emulsifying, encapsulating, entrapping, or lyophilizing processes. The following methods and excipients are merely exemplary and are in no way limiting.

The pharmaceutical compositions may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. Certain organic solvents such as dimethylsulfoxide may also be employed, although usually at the cost of greater toxicity.

For injection, the compounds can be formulated into preparations by dissolving, suspending or emulsifying them in an aqueous or nonaqueous solvent, such as mannitol, 1,3-butanediol, vegetable or other similar oils, aliphatic acid glycerides (e.g., synthetic aliphatic acid glycerides), esters of higher aliphatic acids, or propylene glycol. If desired, the injection preparation may include at least one additive such as solubilizers, isotonic agents (e.g., isotonic sodium chloride solution), suspending agents (e.g., sterile, bland, fixed oils, including synthetic mono- or diglycerides, and fatty acids, including oleic acid), emulsifying agents, stabilizers, and preservatives. For instance, the compounds of the invention may be formulated in aqueous solutions, such as in physiologically compatible buffers such as Hanks's solution, Ringer's solution, or physiological saline buffer.

The compounds may be formulated for parenteral administration by injection, e.g., by bolus injection or continuous infusion. Formulations for injection may be presented in unit dosage form, e.g., in ampules or in multidose containers, with an added preservative. The compositions may take such forms as suspensions, solutions or emulsions in oily or aqueous vehicles, and may contain formulator agents such as suspending, stabilizing, and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Alternatively, the active ingredient may be in powder form for constitution with a suitable vehicle, e.g., sterile pyrogen-free water, before use.

For oral administration, the compounds of formula (I) can be formulated by combining them with known pharmaceutically acceptable carriers. Such carriers enable the compounds to be formulated as tablets, pills, dragees, capsules, emulsions, lipophilic and hydrophilic suspensions, liquids, gels, syrups, slurries, suspensions, and the like, for oral ingestion by a patient to be treated. Pharmaceutical preparations for oral use can be obtained by mixing the compounds with a solid excipient, optionally grinding the resulting mixture, and processing the mixture of granules, after adding suitable auxiliaries, if desired, to obtain tablets or dragee cores. Examples of excipients include, but are not limited to, fillers such as sugars, including lactose, sucrose, mannitol, or sorbitol; and cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid, or a salt thereof such as sodium alginate.

Dragee cores are provided with suitable coatings. The coating may be formed from lacquer solutions or solvent mixtures, such as organic solutions, including concentrated sugar solutions that may optionally contain gum arabic, talc, polyvinyl pyrrolidone, carbopol gel, polyethylene glycol, and/or titanium dioxide. Dyestuffs or pigments may be added to the tablets or dragee coatings for identification or to characterize different combinations of active compound doses.

Oral preparations also include push-fit capsules made of gelatin, as well as soft, sealed capsules made of gelatin and a plasticizer, such as glycerol or sorbitol. The push-fit capsules can contain at least one active ingredient in admixture with filler such as lactose, binders such as starches, lubricants such as talc or magnesium stearate, and/or stabilizers. In soft capsules, the active compounds may be dissolved or suspended in suitable liquids, such as fatty oils, liquid paraffin, or liquid polyethylene glycols. In addition, stabilizers may be added.

When administered orally as a suspension, these compositions may be prepared according to known techniques and may contain bulking agents (e.g., microcrystalline cellulose), suspending agents (e.g., alginic acid, sodium alginate), viscosity enhancers (e.g., methylcellulose), and known sweeteners/flavoring agents. As immediate release tablets, these compositions may contain microcrystalline cellulose, dicalcium phosphate, starch, magnesium stearate, lactose, and/or other excipients, binders, extenders, disintegrants, diluents, and lubricants known in the art.

For buccal administration, the compositions may take the form of tablets or lozenges that may be formulated in a known manner.

For transmucosal administration, penetrants appropriate to the barrier to be permeated may be used in the formulation. Penetrants are known in the art.

For administration by inhalation, the compounds of formula (I) may be delivered in the form of an aerosol spray that may be administered from pressurized packs or a nebulizer, with the use of a suitable propellant, e.g., dichlorodifluoromethane, trichlorofluoromethane, dichlorotetrafluoroethane, carbon dioxide, or other suitable gas, or from propellant-free, dry-powder inhalers. In the case of a pressurized aerosol, the dosage unit may be controlled by providing a valve to deliver a metered amount. Capsules and cartridges of, e.g., gelatin, for use in an inhaler or insufflator may be formulated to contain a powder mix of at least one compound of formula (I) and a suitable powder base such as lactose or starch.

The compounds may also be formulated in rectal compositions such as suppositories or retention enemas, e.g., containing known suppository bases such as cocoa butter, carbowaxes, polyethylene glycols, or other glycerides, all of which melt at body temperature, yet are solid at room temperature.

Additionally, the compounds of formula (I) may be administered via a controlled-release mechanism. If the compounds of this invention are administered via a time-release mechanism or the compounds themselves are altered or modified for controlled-release, their activity and therapeutic effect may be prolonged.

Examples of controlled-release mechanisms suitable for use in the delivery system of the invention include, but are not limited to, controlled-release tablets and capsules, implantable devices, delivery pumps, wafers, biodegradable polymers, topical applications, and combinations thereof. Another example of a controlled-release mechanism is a controlled-release formulation comprising a modified form of the compound, as discussed in more detail below.

Thus, in one aspect, the compounds of formula (I) may be included in controlled-release tablets or capsules. Such tablets and capsules may provide delayed-, modified-, sustained-, or pulsed-release. For example, such capsules or tablets may contain a controlled release formulation, such as a dispersion of active compound in a sustained release material such as glyceryl monostearate, glyceryl distearate, hydroxypropylmethyl cellulose alone, or with a wax. Thus, controlled release of the active ingredient may be achieved by using, for example, hydroxypropylmethyl cellulose in varying proportions to provide the desired release profile.

In addition, the compounds may be delivered via time-release devices. The time-release device may be adapted to deliver the composition at the desired dosing rate with a high degree of precision. The system may protect the composition from degradation by enzymes so that the system does not need to be removed and replaced continuously. The time-release device may be placed just under the skin, for example in the upper arm.

For instance, an osmotic pump may be implanted to continuously deliver drugs at controlled rates. Such pumps can be implanted subcutaneously, intramuscularly, or intraperitoneally, and can be designed for targeted delivery, i.e., to a particular tissue or organ, or for general delivery.

One example of an osmotic pump is the ALZET® pump. These pumps are used in the research setting and are implanted into research animals to maintain certain plasma concentrations of a drug at certain levels. Another example of such a pump is the DUROS® pump. This pump operates like a miniature syringe loaded with a drug inside the drug reservoir. Through osmosis, water from the body is slowly drawn through a semipermeable membrane into the pump by salt residing in the engine compartment. This water fills the pump, which slowly and continuously pushes a piston, dispensing the correct amount of drug out the drug reservoir and into the body. Such osmotic engines do not require batteries, switches, or other electromechanical parts in order to operate. The amount of drug delivered by the system is regulated by the membrane's control over the amount of water entering the pump and by the concentration of the drug in the drug reservoir.

A miniaturized catheter may be attached to an osmotic pump to direct the flow of a drug to the target organ, tissue, or synthetic medical structure, such as a graft. Site-specific delivery enables a therapeutic concentration of a drug to be administered to the desired target without exposing the entire body to a similar dose.

Another controlled-release mechanism for use with the present invention is a wafer. Wafers can be provided in various sizes and various materials and delivered directly to a surgical cavity. For example, during surgery, a wafer may be placed in the cavity. The wafer delivers the active agent directly to the desired site.

In this regard, a plurality of wafers containing the compounds of formula (I) can be implanted in a surgical cavity in order to deliver the compounds of formula (I) to a patient in need thereof. For example, after brain surgery, a wafer containing a compound of formula (I) may be implanted in the surgical cavity. Alternatively, after a mastectomy, a wafer containing at least one compound of formula (I) may be implanted in the surgical cavity.

The compounds of formula (I) may be formulated as a depot preparation. For example, the compounds may be formulated with suitable polymeric or hydrophobic materials (for example as an emulsion in an acceptable oil) or ion exchange resins. The controlled-release mechanism may be a biodegradable matrix or polymer. The composition of this invention can be complexed, mixed with, or otherwise associated with a matrix or polymer that may be injected into or just under a patient's skin. As the matrix or polymer slowly degrades over time, more and more of the active composition can be released. Examples of such matrices or polymers are known in the art.

Although specific controlled-release devices have been described, it is understood that any implantable controlled-release mechanisms that can be used to deliver the compounds of formula (I) is considered within the scope of this invention. For example, implantable delivery devices are also currently used in some patients to deliver birth control over an extended period of time. Such implants or similar implantable devices may be used to deliver the compounds of formula (I).

In another controlled-release aspect of the invention, the compounds may be delivered topically, for example, via a gel or a lotion. When delivered as a topical formula, the compositions may be prepared with any suitable gel or lotion substrate known in the field of topical drug delivery. For example, a gel or lotion may be applied topically, i.e., directly to a patient's skin on or near the site of a skin tumor. In one aspect, the gel or lotion has controlled-release beads or capsules contained therein. However, even without such beads or capsules, the patient may at least partially control the release of the composition by either applying a light layer to the skin or by applying a heavier layer that will take longer to absorb. Additionally or alternatively, a gel or lotion may be applied orally, rectally, nasally, or combinations thereof.

Another topical method involves a patch comprising at least one compound of formula (I). The pharmaceutical composition may be included in a patch for delivery through the skin. Patches of this type are known and may include skin-penetrating enhancers.

A further aspect includes the delivery of the compounds of formula (I) via iontophoresis. Specifically, the number of drugs that may be delivered by the transdermal route is limited by the barrier properties of the skin. Conventional transdermal therapy is traditionally limited to small, potent, lipophilic drugs. Iontophoresis is one strategy that facilitates transdermal drug delivery. Iontophoresis is facilitated movement of ions across a membrane, e.g., the skin, in order to deliver a positively charged drug across the skin. For example, a solution of a cationic formulation of the compound of formula (I) may be placed at the positive electrode where it is repelled and then attracted toward a negative electrode placed elsewhere on the body.

Another controlled-release mechanism for use with this invention comprises a controlled-release form of the compound itself. For instance, the compound may be a sparingly soluble salt. Alternatively, the controlled-release mechanism may comprise a modification or alteration of the compound itself. For example, the controlled-release mechanism may comprise modified forms of the compounds of formula (I) that are chemically changed, encapsulated, caged, protected, lipidized, structurally modified to enhance stability, glycosylated, combined with nutrient transporters, used as a prodrug, incorporated with vector-based strategies, cationized, conjugated with a polymer, or combinations thereof, such that the compounds are at least partially altered from the structures described above. The modification may enhance the compound's permeability through a patient's blood-brain barrier, may enhance delivery, e.g., by targeting the compound to tumors, may allow later activation, and combinations thereof.

Modifying the compounds of formula (I) by receptor-mediated mechanisms includes altering the compounds of formula (I) or packaging them into an agent that is capable of binding to tumor cell receptors and therefore selectively entering tumor cells. This allows specific targeting of these agents to tumor cells with a low incidence of side effects since the drug only enters cells with appropriate receptors.

One aspect of this invention relates to compounds of formula (I) modified with tumor specific antibodies, ligands for tumor specific proteins, or as an adduct for the compound for tumor targeting purposes. For example, tumor associated antigens (“TAA”) are highly, homogeneously, frequently, and selectively expressed on the cell surface in clinical tumor samples and represent potentially excellent targets for tumor immunotherapy. The use of antibodies selective for TAA or other ligands for tumor specific proteins added to the structures of the compounds of formula (I) is believed to enable the precise targeting of those agents to tumor tissue.

Monoclonal antibodies may be optionally conjugated to liposomes and directed against a tumor marker. TNF-α or a TNF-α receptor may be employed. In addition, targeting of a marker to abnormal tumor vasculature can be employed. The targeting moiety when coupled to a toxic drug or radioisotope will act to concentrate the drug where it is needed. Ligands for tumor-associated vessel markers can also be used. For example, a cell adhesion molecule that binds to a tumor vascular element surface marker can be employed.

Examples of targeting strategies include, but are not limited to, ADEPT (antibody-directed enzyme prodrug therapy), GDEPT (gene-directed EPT), and VDEPT (virus-directed EPT). Other strategies include targeting differentially expressed genes, enzymes, or surface markers that appear on tumor-associated vasculature, to effect control of tumor growth.

ADEPT is described in UCHEGBU, Pharmaceutical Journal, 263(7061):355-358 (1999). The principle behind the ADEPT approach is that an antibody-enzyme conjugate is administered intravenously, localizes in tumor tissue, and subsequently activates an administered prodrug predominantly within such tumors. Prodrug activation occurs on the cell surface or in the extracellular fluid. The enzyme milieu in or about the tumor transforms the prodrug into an active agent that then acts on the tumor tissue. Similarly, differential gene expression or viral targeting at the tumor site is used to activate a prodrug into its active, toxic form in GDEPT and VDEPT, respectively.

The appearance of the active drug after prior administration of the antibody-enzyme conjugate to patients confirms the feasibility of the ADEPT approach. In some instances, to promote specificity, a non-human, e.g., bacterial, enzyme such as carboxypeptidase G2 is used to activate the prodrug. Because such enzymes may also elicit an immune response, it may also be necessary to administer an immunosuppressant. Preferably, the prodrug should be nontoxic and the enzyme should locate only at tumor sites.

Another example of prodrugs are polymeric prodrugs. UCHEGBU describes the use of polymeric prodrugs (commonly known as polymer drug conjugates), which involves the use of an active substance and possibly a targeting moiety, both linked via spacers to a water-soluble polymeric backbone. From this basic configuration, a number of polymer drug conjugates for cancer chemotherapy have been synthesized with cleavable drug polymer linkers.

Polymer drug conjugates accumulate selectively within tumor tissue and leak through the disorganized vasculature. Clearance from tumor tissue is delayed due to the poor lymphatic drainage, thus tumor accumulation of polymer drug conjugates has been called the enhanced permeation and retention effect. On IV administration the conjugate is taken up by tumor cells, and the active drug is released intracellularly.

Most of the polymeric backbones that have been studied are prepared from non-biodegradable materials. Although in some instances, biodegradable polymers may be more acceptable, care must be taken to ensure that biodegradation does not hamper the accumulation of conjugates in tumor tissue.

The use of polymer drug conjugates is believed to improve the activity of anticancer agents. Polymer drug conjugates also decrease distribution to potential sites of toxicity. By targeting certain compounds away from sites of potential toxicity, polymer conjugates can significantly increase the maximum tolerated dose of a compound in patients.

Liposomes and other drug delivery systems can also be used, especially if their surface contains a ligand to direct the carrier preferentially to, e.g., tumor vasculature. Liposomes readily permeate lipid rich cell membranes and enhance delivery to cells. Liposomes are formed by the self-assembly of phospholipid molecules in an aqueous environment. The amphipathic phospholipid molecules form a closed bilayer sphere in an attempt to shield their hydrophilic groups from the aqueous environment, while still maintaining contact with the aqueous phase via the hydrophilic head group. The resulting closed sphere may encapsulate the compounds of formula (I) with the bilayer membrane.

Liposomes offer the added advantage of shielding the drug from most normal tissues, thereby reducing the inherent toxicity of many compounds. When coated with polyethylene glycol (PEG) (i.e., stealth liposomes) to minimize uptake by phagocytes and with a tumor-targeting moiety, liposomes offer longer plasma half-lives, lower non-target tissue toxicity, and increased efficacy over non-targeted drug. See, e.g., U.S. Pat. No. 5,013,556 to WOODLE et al.

Alternatively, lipid soluble drugs may be complexed with cyclodextrines and subsequently encapsulated within the liposome aqueous compartment. Drug encapsulation within or associated with liposomes in this way alters drug pharmacokinetics and may be useful in various targeted therapies. These concepts may be used in conjunction with compounds of formula (I) in order to optimize liposomal drug targeting and delivery. For example, the reduced liver and spleen uptake of stealth liposomes is believed to be due to a reduced coating recognition by the liver and spleen.

In yet another aspect of this invention, the controlled-release mechanism for the compounds of formula (I) enhances the permeability of the composition through a patient's blood-brain barrier in order to treat brain tumors. This may be done through any number of methods. Examples include, but are not limited to, disruption of the blood brain barrier, receptor-mediated mechanisms, and liposomal encapsulation.

For example, it has been reported that temporarily opening the blood-brain barrier can allow chemotherapeutic agents to pass into the brain and reach the tumor. See, e.g., www.ohsu.edu/hosp-bbb/bbbdtherapy.html. Specifically, the brain's protective barrier is composed of tightly knit endothelial cells, which line the walls of the blood vessels in the brain. These tightly knit cells create a barrier that blocks the entry of various substances, including many therapeutic agents. By temporarily shrinking these cells with a concentrated sugar solution, the barrier can be opened, allowing chemotherapy drugs, such as the compounds of formula (I), to pass into the brain and reach the tumor. It has been found that compared with standard chemotherapy, blood-brain barrier disruption therapy increases the delivery of the chemotherapy drugs to the tumor and its surrounding area around the tumor by tenfold to a hundredfold.

Another aspect of the invention includes using peptide drug transporters linked to, or otherwise associated with, compounds of formula (I) to enhance access to the brain. It has been found that various peptide drug modifications can enhance bioavailability and blood-brain barrier permeability. See, e.g., WITT et al., Peptides, 22:2329 (2001). This article discusses modifications such as lipidization, structural modification to enhance stability, glycosylation, use of nutrient transporters, prodrugs, vector-based strategies, cationization, and polymer conjugation.

Using the foregoing techniques, the compounds of formula (I) can be targeted to tumors and tumor vasculature to effect control of tumor progression or to other sites of interest (e.g., endothelial cells).

According to the methods of the present invention, compounds of formula (I) can be administered alone, in combination with other compounds of formula (I), or in combination with other known compounds suitable for treating or ameliorating the effects of cancer and/or the treatment thereof (e.g., other anti-cancer drugs or other drugs, such as AZT, anti-inflammatories, antibiotics, corticosteroids, vitamins, etc.).

For instance, the compounds of formula (I) can be used in conjunctive therapy with other known anti-angiogenic, chemotherapeutic, or antineoplastic agents (e.g., vinca alkaloids, antibiotics, antimetabolites, platinum coordination complexes, etc.). According to certain embodiments, the compounds of formula (I) can be used in conjunctive therapy with a vinca alkaloid compound, such as vinblastine, vincristine, taxol, etc.; an antibiotic, such as adriamycin (doxorubicin), dactinomycin (actinomycin D), daunorubicin (daunomycin, rubidomycin), bleomycin, plicamycin (mithramycin), mitomycin (mitomycin C), etc.; an antimetabolite, such as methotrexate, cytarabine (AraC), azauridine, azaribine, fluorodeoxyuridine, deoxycoformycin, mercaptopurine, etc.; or a platinum coordination complex, such as cisplatin (cis-DDP), carboplatin, etc. Other classes of chemotherapy include but are not limited to: covalent DNA binding drugs, DNA-based topoisomerase inhibitors (I and II), differentiation agents, hormonal agents, enzymes, and any combination thereof. Further examples and descriptions are provided in PERRY, Chemotherapy Source Book, 2d ed. (1997).

In another aspect, the compounds of formula (I) may be delivered in combination with other tumor or cancer therapies. For example, the compounds of formula (I) may be used as a preventive measure after surgical excision or in combination with other anti-cancer treatments. For instance, the compounds of formula (I) may be delivered in combination with radiation therapy, phototherapy, surgical resection, immunotherapy, vaccination, interferon treatment, stereotactic surgery, such as Gamma Knife® surgery, and combinations thereof.

The above treatments are, of course, merely provided as examples and are not intended to be exhaustive of the possible treatments available and not intended to limit the present invention. It is anticipated that cancer researchers will invent and/or discover other therapies that may be used to treat tumors, and the compounds of formula (I) used in combination with such treatments to treat neoplastic diseases is considered within the scope of this invention.

Pharmaceutical compositions suitable for use in the methods of the present invention include compositions wherein the active ingredients are contained in a therapeutically effective amount. The amount of composition administered will, of course, be dependent on the subject being treated, on the subject's weight, the severity of the affliction, the manner of administration, and the judgment of the prescribing physician. Determination of an effective amount is well within the capability of those skilled in the art, especially in light of the detailed disclosure provided herein.

For any compound used in the method of the invention, a therapeutically effective dose can be estimated initially from cell culture assays. For example, a dose can be formulated in animal models to achieve a circulating concentration range that includes the EC₅₀ as determined in cell culture (i.e., the concentration of test compound that is lethal to 50% of a cell culture), or the EC₁₀₀ as determined in cell culture (i.e., the concentration of compound that is lethal to 100% of a cell culture). Such information can be used to more accurately determine useful doses in humans. Initial dosages can also be estimated from in vivo data.

Moreover, toxicity and therapeutic efficacy of the compounds described herein can be determined by standard pharmaceutical procedures in cell cultures or experimental animals, e.g., by determining the LD₅₀, (the dose lethal to 50% of the population) and the ED₅₀ (the dose therapeutically effective in 50% of the population). The dose ratio between toxic and therapeutic effect is the therapeutic index and can be expressed as the ratio between LD₅₀ and ED₅₀. Compounds that exhibit high therapeutic indices are preferred. The data obtained from these cell culture assays and animal studies can be used in formulating a dosage range that is not toxic for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration, and dosage can be chosen by the individual physician in view of the patient's condition. See, e.g., FINGL et al., The Pharmacological Basis of Therapeutics (1975).

Doses and dosage levels will vary depending upon the state of the condition treated, the delivery route chosen, and other physical considerations. In this regard, the dosage amount and interval may be adjusted individually to provide plasma levels of the active compound that are sufficient to maintain a therapeutic effect. Usual patient dosages for oral administration range from about 50-2000 mg/kg/day, commonly from about 100-1000 mg/kg/day, such as from about 150-700 mg/kg/day and most preferably from about 250-500 mg/kg/day. For instance, the dosage may range from about 0.02 to about 10.0 g/day, such as from 0.1 to 5.0 g/day, which has been found useful in the treatment or prevention of the above-indicated conditions, with oral doses two-to-five times higher. The therapeutically effective serum levels may be achieved by administering multiple doses each day.

In cases of local administration or selective uptake, the effective local concentration of the drug may not be related to plasma concentration. One having skill in the art will be able to optimize therapeutically effective local dosages.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The present invention will be further illustrated by way of the following Examples. These Examples are non-limiting and do not restrict the scope of the invention.

### EXAMPLES

### Example 1

### Alkoxide Addition

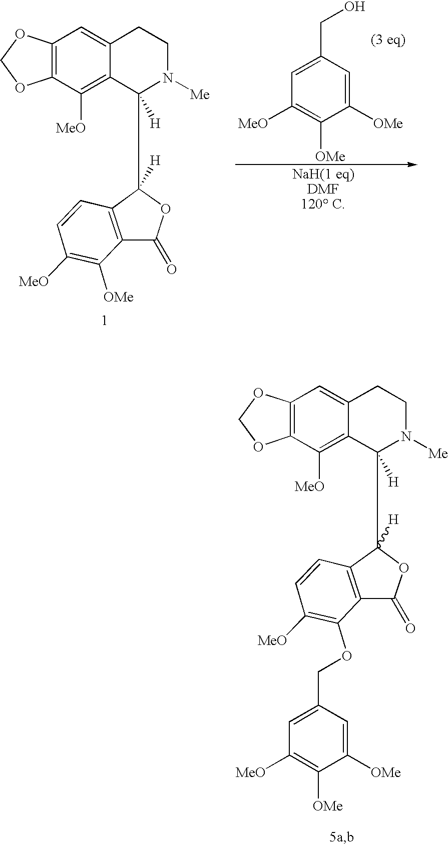

Sodium hydride (19 mg, 0.48 mmol, Aldrich) was added to a mixture of (S,R)-noscapine 1 (200 mg, 0.48 mmol, Aldrich) and 3,4,5-trimethyoxybenzyl alcohol (233 μL, 1.45 mmol, ACROS) in DMF (1.0 mL). The resulting mixture was stirred vigorously at room temperature for 5 min, heated to 120° C. for 15 min, and cooled to room temperature. Aqueous HCl (6 M, 0.5 mL) was added, and the mixture was extracted with CH₂Cl₂ (3×5 mL). The CH₂Cl₂ layers were combined and concentrated. The crude product was purified by reversed-phase HPLC to give an approximate 1:1 mixture of diastereomers 5a and 5b.

HPLC Conditions:

Column: Waters XTerra Preps Column (Cl₈, 5 μm, 19×50 mm)

Solvent conditions: A: 10 mM aq NH₄OAc (pH 5.75)
B: CH₃CN
Gradient 0-7 min: 90% A 10% B
7-9 min: 1% A 99% B
9-10 min: 90% A 10% B

Flow Rate: 10 mL/min

Detection: 254 and 220 nm

Retention time of 5a,b (both diastereomers): 6.6 min

### Example 2

### Alkoxide Addition

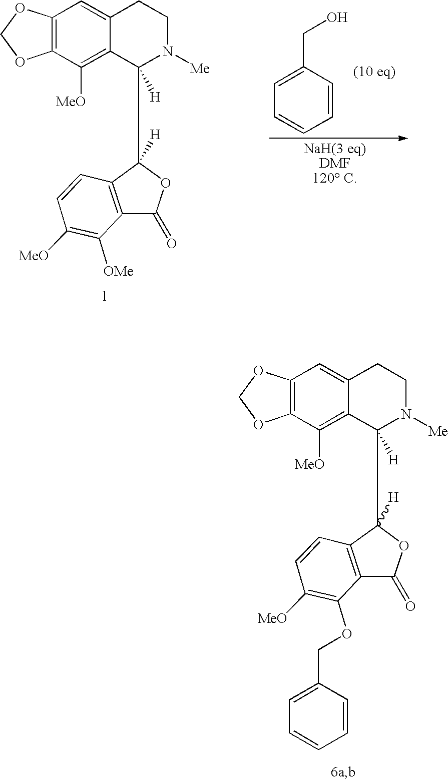

(S,R)-Noscapine 1 (10 g, 24 mmol, Aldrich) was added to a stirred mixture of benzyl alcohol (25 mL, 240 mmol, Aldrich) and sodium hydride (2.9 g, 73 mmol, Aldrich) in DMF (15 mL). The resulting mixture was heated to 120° C. for 16 h and then cooled to room temperature. Aqueous HCl (6 M) was added until pH˜3, and the mixture was extracted with ethyl acetate (3×100 mL). The ethyl acetate layers were combined, dried over Na₂SO₄, filtered, and concentrated. The crude product was purified by column chromatography (60% ethyl acetate-hexanes) to give 1.6 g of compounds 6a,b (14%) as a 1:1 mixture of diastereomers. The two diastereomers were separated using reversed-phase HPLC (Waters NovaPak C₁₈ column, 19×300 mm; 70:30 CH₃CN-10 mM aq NH₄OAc (pH 5.75), 10 mL/min)

Diastereomer 6a: (Retention time: 16.1 min) ¹H NMR (CDCl₃, 300 MHz): δ 7.58-7.52 (m, 2H), 7.37-26 (m, 3H), 7.12 (d, J=8.2 Hz, 1H), 6.95 (d, J=8.1 Hz, 1H), 6.35 (s, 1H), 5.89-5.84 (m, 2H), 5.51 (s, 1H), 5.37 (s, 2H), 4.22 (brs, 1H), 3.95 (s, 3H), 3.86 (s, 3H), 3.18-3.03 (m, 1H), 2.88-2.72 (m, 1H), 2.72-2.43 (m, 2H), 2.11 (s, 3H). ¹³C NMR (CDCl₃, 300 MHz): δ 152.7,139.9, 137.1, 133.7, 128.6, 128.2, 128.0, 118.7, 116.9, 102.4, 100.6, 76.1, 61.1, 59.1, 56.9, 49.7, 45.0, 26.7. LC-Mass (ES): [M+1]⁺ calculated for C₂₈H₂₈NO₇, 490.18; found, 490.24.

Diastereomer 6b: (Retention time: 17.9 min) ¹H NMR (CDCl₃, 300 MHz): δ 7.57-7.50 (m, 2H), 7.37-7.21 (m, 3H), 6.90 (d, J=8.2 Hz, 1H), 6.28 (s, 1H), 6.07-5.96 (m, 1H), 5.92 (dd, J₁=1.4 Hz, J₂=2.4 Hz, 2H), 5.57 (d, J=3.4 Hz, 1H), 5.35 (s, 2H), 4.40 (d, J=3.9 Hz, 1H), 4.02 (s, 3H), 3.80 (s, 3H), 2.60-2.50 (m, 4H), 2.38-2.24 (m, 2H), 1.87-1.71 (m, 1H). ¹³C NMR (CDCl₃, 300 MHz): δ 168.1, 152.7, 148.4, 146.0, 140.9, 140.4, 137.1, 134.0, 128.8, 128.1, 127.9, 118.2, 117.9, 102.3, 100.8, 81.7, 76.0, 60.8, 59.4, 56.8, 50.0, 46.4, 28.1. LC-Mass (ES): [M+1]⁺ calculated for C₂₈H₂₈NO₇, 490.18; found, 490.24.

### Example 3

### Selective Dealkylation

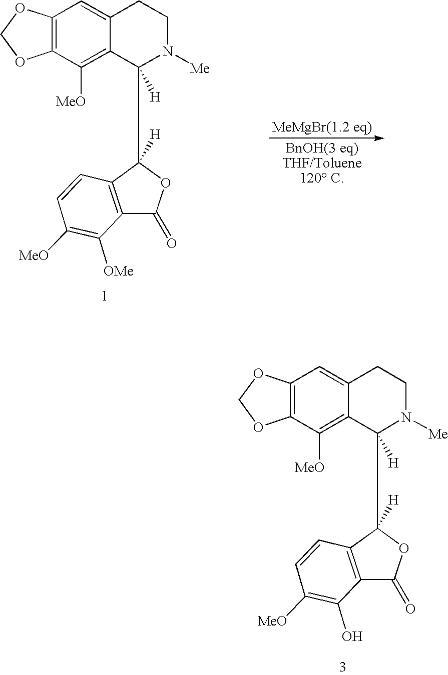

Benzyl alcohol (3.8 mL, 36.3 mmol, Aldrich) was slowly added to MeMgBr (10.4 mL of 1.4 M in THF-toluene, 14.5 mmol, Aldrich) at 0° C., and the mixture was stirred for 5 min. (S,R)-Noscapine (5 g, 12.1 mmol, Aldrich) was added as a solid, and the resulting mixture was heated to 100° C. for 2 h and then to 120° C. for 3 h. The reaction mixture was cooled to room temperature, diluted with 1 M aq HCl (30 mL) and H₂O (40 mL), and then extracted with diethyl ether (3×30 mL) to remove benzyl alcohol. The aqueous layer was basified to pH˜8 using saturated aq NaHCO₃ and then extracted with ethyl acetate (3×100 mL). The ethyl acetate layers were combined and washed with saturated aq NaCl (75 mL), dried over Na₂SO₄, decanted, and concentrated to give 2.31 g of crude product consisting of approximately 60% of the desired phenol 3.

¹H NMR of a purified (SiO₂ chromatography using 40-50% ethyl acetate gradient) sample (DMSO-d₆, 300 MHz): δ 9.75 (s, 1H), 7.10 (d, J=8.1 Hz, 1H), 6.46 (s, 1H), 5.98-6.03 (m, 2H), 5.81 (d, J=8.1 Hz, 1H), 5.48 (d, J=3.8 Hz, 1H), 4.23 (d, J=4.1 Hz, 1H), 3.96 (s, 3H), 3.79 (s, 3H), 2.48-2.60 (m, 1H), 2.34-2.47 (m+s, 4H), 2.19-2.32 (m, 1H), 1.85-1.97 (m, 1H). ¹³C NMR (DMSO-d₆, 300 MHz): δ 167.8, 148.0, 147.4, 145.3, 140.0, 133.9, 131.6, 117.4, 116.5, 113.4, 112.5, 102.3, 100.9, 80.7, 60.5, 59.3, 56.6, 49.1, 45.7, 26.9. LC-Mass (ES): [M+1]⁺ calculated for C₂₁H₂₁NO₇, 400.14; found, 400.34.

### Example 4

### Direct Alkylation

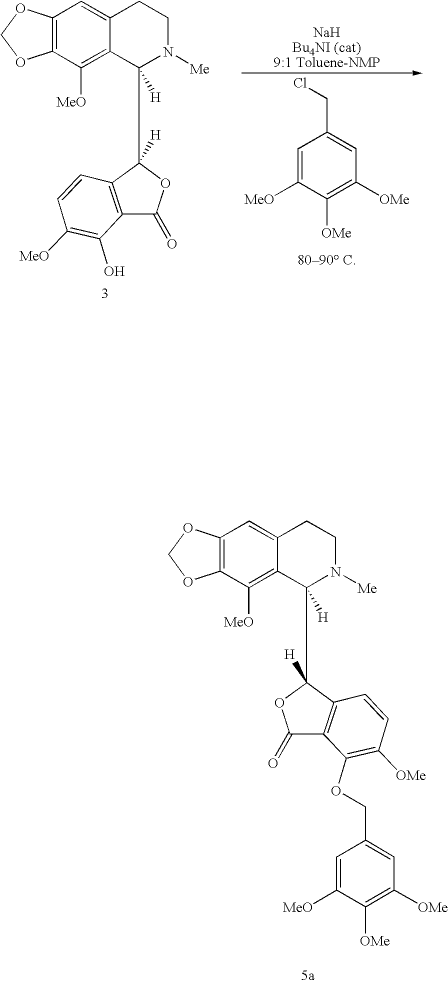

NaH (0.28 g (60 wt % dispersion in oil), 6.94 mmol, Aldrich) was added to a stirred solution of 7-hydroxy noscapine 3 (2.31 g, 5.78 mmol) in toluene (25 mL) and 1-methyl-2-pyrrolidinone (3 mL) over a period of 3 min at room temperature. 3,4,5-Trimethoxybenzyl chloride (1.88 g, 8.78 mmol) was then added, followed by a catalytic amount of tetrabutylammonium iodide (ca. 25 mg, Aldrich). The resulting solution was heated to 80° C. and stirred overnight (ca. 18 h). LC-Mass confirmed the complete consumption of compound 3. The reaction mixture was cooled to room temperature, diluted with H₂O (40 mL), and extracted using ethyl acetate (3×75 mL). The ethyl acetate layers were combined and dried over Na₂SO₄, decanted, and concentrated. The crude product was purified using column chromatography (SiO₂) and reversed-phase HPLC (Waters NovaPak C₁₈ column, 19×300 mm; 60:40 CH₃CN-10 mM aq NH₄OAc (pH 5.75), 10 mL/min) to give compound 5a (1.52 g, 22% yield based on compound 3, retention time: 22.5 min).

¹H NMR (DMSO-d₆, 300 MHz): δ 7.26 (d, J=8.3 Hz, 1H), 6.85 (s, 2H), 6.44 (s, 1H), 6.10 (d, J=8.3 Hz, 1H), 5.98-6.03 (m, 2H), 5.53 (d, J=4.3 Hz, 1H), 5.18 (dd (apparent q), J=16.4 Hz, 2H), 4.23 (d, J=4.3 Hz, 1H), 3.93 (s, 3H), 3.84 (s, 3H), 3.76 (s, 6H), 3.63 (s, 3H), 2.47-2.33 (s+m, 5H), 2.27-2.16 (m, 1H), 1.83-1.70 (m, 1H)

¹³C NMR (DMSO-d₆, 300 MHz): δ 167.4, 152.6, 152.2, 148.0, 140.4, 140.0, 133.9, 132.7, 131.4, 119.5, 118.9, 117.8, 116.4, 105.0, 102.4, 100.8, 80.7, 74.8, 60.4, 59.9, 59.2, 56.6, 55.7, 48.8, 45.5, 26.6. LC-Mass (ES): [M+1]⁺ calculated for C₃₁H₃₄NO₁₀, 580.21; found, 580.39.

### Example 5

### Direct Alkylation

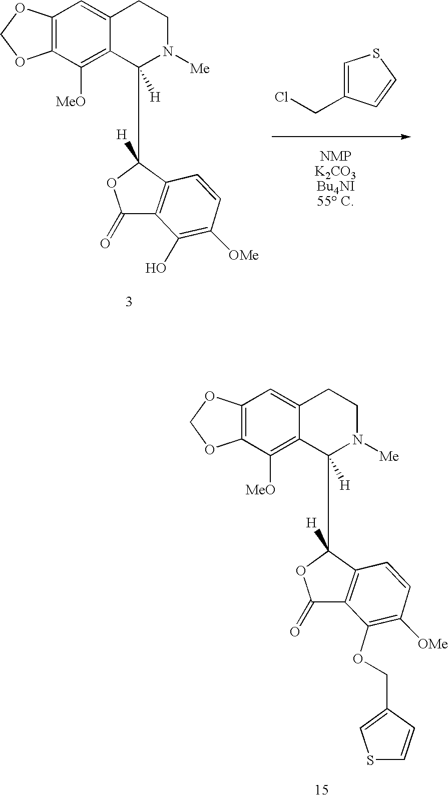

A mixture of the phenol (37.5 mg), alkyl chloride (19 mg), potassium carbonate (39 mg), and tetrabutylammonium iodide (5 mg) in 1-methyl-2-pyrrolidinone (500 μL) was heated to 55° C. for 17 h. The reaction mixture was cooled and diluted with ethyl acetate (10 mL) and H₂O (3 mL). The H₂O layer was extracted with ethyl acetate (3 mL), and the ethyl acetate layers were combined and concentrated. The concentrate was purified by Prep. LC-Mass to give the desired product. LC-Mass (ES): [M+1]⁺ calculated for C₂₆H₂₆NO₇S, 496.14; found, 496.29.

### Example 6

### Direct Alkylation

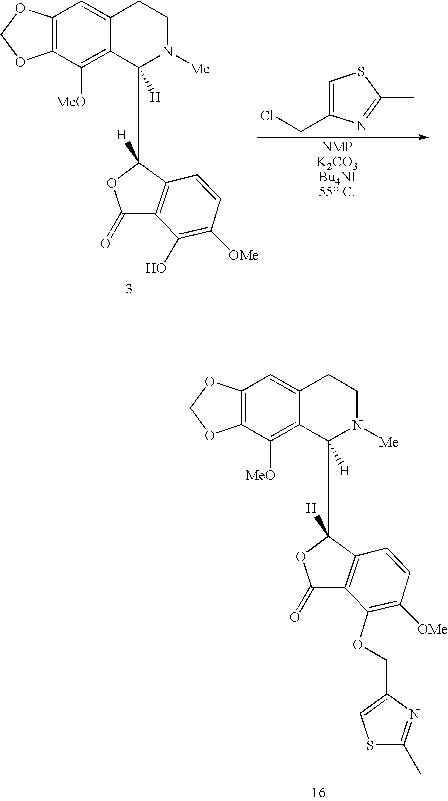

A mixture of the phenol (49.1 mg), 4-(chloromethyl)-2-methyl-1,3-thiazole (27 mg), potassium carbonate (51 mg), and tetrabutylammonium iodide (5 mg) in 1-methyl-2-pyrrolidinone (500 μL) was heated to 55° C. for 17 h. The reaction mixture was cooled and diluted with ethyl acetate (10 mL) and H₂O (3 mL). The H₂O layer was extracted with ethyl acetate (3 mL), and the ethyl acetate layers were combined and concentrated. The concentrate was purified by Prep. LC-Mass to give the desired product. LC-Mass (ES): [M+1]⁺ calculated for C₂₆H₂₇N₂O₇S, 511.15; found, 511.17.

### Example 7

### N-alkylation

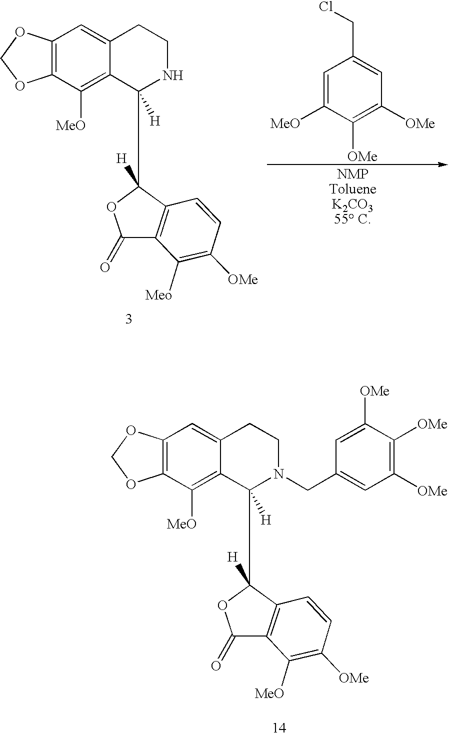

A mixture of N-demethylated noscapine (22.0 mg), 3,4,5-trimethoxybenzyl chloride (17.9 mg), and potassium carbonate (50 mg) in 1-methyl-2-pyrrolidinone (100 μL) and toluene (200 μL) was heated to 55° C. for 18 h. The reaction mixture was cooled and diluted with ethyl acetate (10 mL) and H₂O (5 mL). The H₂O layer was washed with ethyl acetate (5 mL), and the ethyl acetate layers were combined and concentrated. The concentrate was purified by Prep. LC-Mass to give the desired product. LC-Mass (ES): [M+1]⁺ calculated for C₃₁H₃₄NO₁₀, 580.21; found, 580.39.

### Example 8

### Preparation of 7-amino-6-methoxy-3-(4-methoxy-6-methyl-5,6,7,8-tetrahydro-[1,3]dioxolo[4,5-g]isoquinolin-5-yl)-3H-isobenzofuran-1-one

As in more detail below, compound 9a,b was formed from 7-hydroxy noscapine 3 using the following reaction scheme. 7-hydroxy noscapine may be prepared by the method of SCHMIDHAMMER et al., Arch. Pharm., 311:664-671 (1978).

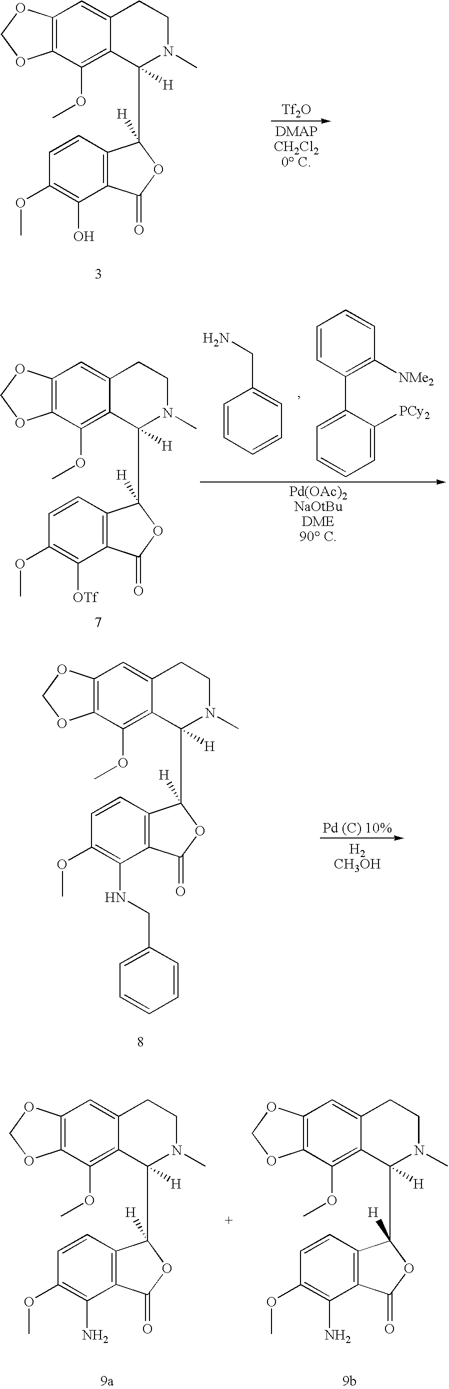

### Step 1. Preparation of trifluoromethanesulfonic acid 5-methoxy-3-(4-methoxy-6-methyl-5,6,7,8-tetrahydro-[1,3]dioxolo[4,5-g]isoquinolin-5-yl)-3-oxo-1,3-dihydro-isobenzofuran-4-yl (compound 7):

A solution of 7-hydroxy noscapine 3 (7-hydroxy-6-methoxy-3-(4-methoxy-6-methyl-5,6,7,8-tetrahydro-[1,3]dioxolo[4,5-g]isoquinolin-5-yl)-3H-iso-benzofuran-1-one) (200 mg, 0.5 mmol) and dimethylaminopyridine (225 mg, 17.5 mmol) in CH₂Cl₂ (10 mL) was treated with triflic anhydride (162 mg, 0.5 mg) dropwise at 0° C. over 1 h. The reaction was stirred at 0° C. for 1 h and washed with 1N HCl (5 mL) and H₂O (5 mL). The organic layer was dried over MgSO₄ and concentrated in vacuo. The resulting residue was purified via flash silica gel chromatography (10%-60%, ethyl acetate/hexane) to provide compound 7 (110 mg, 41% yield). MS (ES): [M+1]⁺, calculated for C₂₂H₂₁F₃NO_{g}S, 532.08; found, 532.68. RP-HPLC analysis (Water XTerra C18 column, 4.6×50 mm, 10-90% CH₃CN/H₂O, 5 min): retention time 3.99 min.

### Step 2. Preparation of 7-benzylamino-6-methoxy-3-(4-methoxy-6-methyl-5,6,7,8-tetrahydro-[1,3]dioxolo[4,5-g]isoquinolin-5-yl)-3H-isobenzofuran-1-one (compound 8):

A dry vial was charged with compound 7 (531 mg, 0.1 mmol), palladium acetate (11 mg, 0.05 mmol), and (2′-dicyclohexylphosphanyl-biphenyl-2-yl)-dimethyl-amine (39 mg, 0.1 mmol) and then evacuated. The vial was filled with argon. Then, methyleneglycol dimethyl ether (anhydrous) (10 mL), benzylamine (160 mg, 1.5 mmol), and sodium tert-butoxide (144 mg, 1.5 mmol) were added, and the vial was sealed. The reaction was stirred at 90° C. overnight, then diluted with ethyl acetate and washed with H₂O. The organic layer was dried over MgSO₄ and concentrated in vacuo. The residue was purified via flash chromatography (5% -40%, ethyl acetate/hexane) to provide compound 8 (110 mg, 45% yield). ¹H NMR (DMSO-d₆, 300MHz): δ 7.28 (m, 6H), 6.96 (d, J=8.4 Hz, 1H), 6.45 (m, 2H), 5.98 (s, 2H), 5.48 (s, 1H), 4.90 (m, 2H), 4.2 (s, 1H), 3.90 (d, 3H), 3.75 (s, 3H), 2.61-2.5 (m, 2H), 2.42 (s, 3H), 2.24 (m, 1H), 1.94 (m, 1H). MS (ES): [M+1]⁺ calculated for C₂₈H₂₉N₂O₆, 489.19; found, 489.79. RP-HPLC analysis (Water XTerra C18 column, 4.6×50 mm, 10-90% CH₃CN/H₂O, 10 min): retention time 5.01 min.

### Step 3. Preparation of 7-amino-6-methoxy-3-(4-methoxy-6-methyl-5,6,7,8-tetrahydro-[1,3]dioxolo[4,5-g]isoquinolin-5-yl)-3H-isobenzofuran-1-one (compound 9):

Compound 8 (100 mg, 0.2 mmol) and palladium on carbon (10%, 5 mg) were added to a flask charged with CH₃0H (8 mL). The resulting mixture was stirred under a hydrogen balloon at room temperature overnight. The catalyst was filtered off, and the filtrate was concentrated in vacuo. The residue was purified via Prep. LC-Mass (Water XTerra C18 column, 19×50 mm, 10-99% CH₃CN/H₂O in 10 min.).

Compound 9a: ¹H NMR (DMSO-d₆, 300 MHz): δ 6.86 (d, J=9.4 Hz, 1H), 6.40 (s,1H), 6.00 (s, 2H), 5.74 (s, 2H), 5.53 (d, J=8.4 Hz,1H), 5.48 (d, J=4.0 Hz, 1H), 4.22 (d, J=4.0 Hz, 1H), 3.90 (s, 3H), 3.75 (s, 3H), 2.61-2.5 (m, 2H), 2.42 (s, 3H), 2.24 (m, 1H), 1.94 (m, 1H). MS (ES): [M+1]⁺ calculated for C₂₁H₂₃N₂O₆, 399.15; found: 399.16. RP-HPLC analysis (Water XTerra C18 column, 4.6×50 mm, 10-90 % CH₃CN/H₂O, 10 min): retention time 3.11 min.

Compound 9b: ¹H NMR (DMSO-d₆, 300 MHz): δ 7.10 (d, J=7.5 Hz, 1H), 6.60 (d, J=7.5 Hz, 1H), 6.47 (s, 1H), 5.94 (s, 2H), 5.75 (s, 2H), 5.43 (s, 1H), 4.13 (s, 1H), 3.95 (s, 3H), 3.82 (s, 3H), 2.99-2.88 (m, 2H), 2.55 (br s, 2H), 2.39-2.32 (m, 1H), 2.07 (s, 3H). MS (ES): [M+1]⁺ calculated for C₂₁H₂₃N₂O₆, 399.15; found: 399.26. RP-HPLC analysis (Nova-Pak C18 column, 19×300 mm, 10-90% CH₃CN/H₂O, 16 min): retention time 12.80 min.

### Example 9

### 5-methoxy-1-(4-methoxy-6-methyl-5,6,7,8-tetrahydro-[1,3]dioxolo-[4,5-g]isoguinolin-5-yl)-3-oxo-1,3-dihydro-isobenzofuran-4-carbonitrile

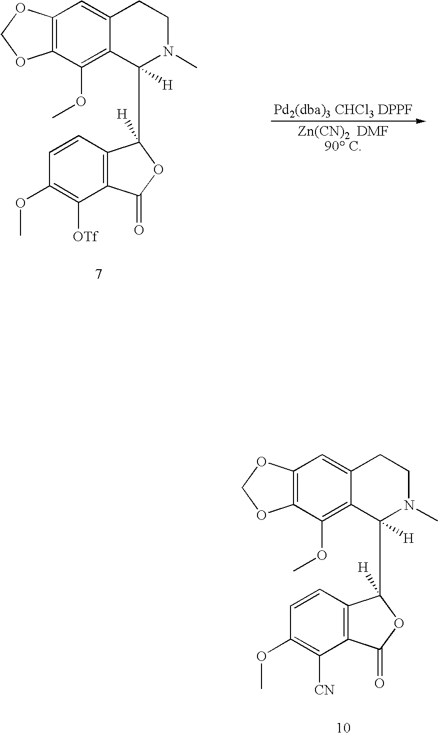

Compound 7 (53.1 mg, 0.1 mmol) was added to a mixture of tris(dibenzylideneacetone) dipalladium chloroform adduct (Pd₂(dba)₃ CHCl₃) (3.7 mg, 0.04 mmol) and 1,1′-bis(diphenylphosphino)ferrocene(DPPF) (8.9 mg, 0.08 mmol) in DMF (1 mL). The resulting mixture was heated to 90° C., and zinc cyanide (11.1 mg, 0.11 mmol) was added over 1 h. The reaction was stirred at 90° C. overnight, then diluted with ethyl acetate (3 mL) and washed with H₂O. The organic layer was washed with brine, dried over MgSO₄, and concentrated in vacuo. The residue was purified via LC-Mass (Water XTerra C18 column, 19×50 mm, 25-99% CH₃CN/H₂O in 10 min) to provide compound 10 (13 mg, 32% yield). ¹H NMR (DMSO-d₆, 300MHz): δ 7.55 (d, J=7.0 Hz, 1H), 6.69 (d, J=9.0 Hz, 1H), 6.48 (s,1H), 6.00 (d, J=2.2 Hz, 2H), 5.70 (d, J=3.7 Hz, 2H), 4.30 (d, J=3.7 Hz 1H), 3.96 (s, 6H), 2.55 (m, 2H), 2.49 (s, 3H), 2.27 (m, 1H), 1.77 (m, 1H). MS (ES): [M+1]⁺ calculated for C₂₂H₂₁N₂O₆: 409.13; found, 409.17. RP-HPLC analysis (Water XTerra C18 column, 4.6×50 mm, 10-90% CH₃CN/H₂O, 10 min): retention time 3.34 min.

### Example 10

### Preparation of 7-(3,5-difluoro-phenyl)-6-methoxy-3-(4-methoxy-6-methyl-5,6,7.8-tetrahydro-[1,3]dioxolo[4,5-q]isoguinolin-5-yl)-3H-isobenzofuran-1-one

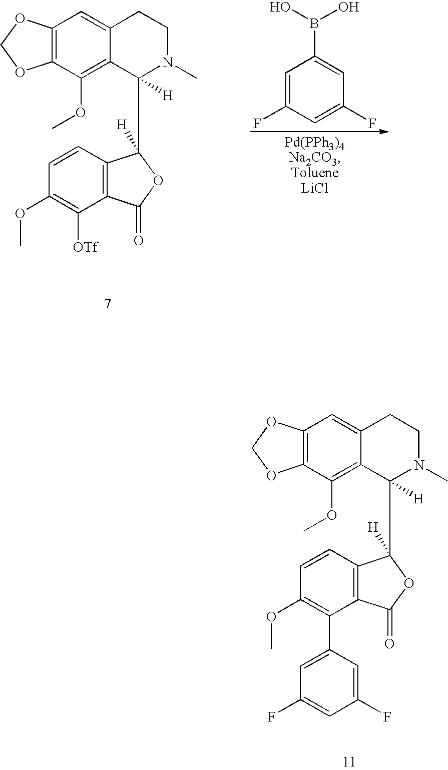

A vial was charged with compound 7 (50 mg, 0.09 mmol), 3,5-difluorophenylboronic acid (22.9 mg, 0.18 mmol), tetrakis(triphenylphosphine) palladium (5.7 mg, 0.0047 mmol), 2.0 M aqueous solution of sodium carbonate (0.1 mL, 0.18 mmol), lithium chloride (8.5 mg, 0.18 mmol), and toluene (1 mL). The vial was filled with argon and sealed, and the reaction was stirred at 80° C. overnight, then diluted with ethyl acetate (3 mL) and washed with H₂O. The organic layer was washed with brine, dried over MgSO₄, and concentrated in vacuo. The residue was purified via LC-MS (Water XTerra C18 column, 19×50 mm, 25-99% CH₃CN/H₂O in 10 min.) to provide compound 11 (23 mg, 50% yield).

¹H NMR (DMSO-d₆, 300 MHz): δ 7.37 (d, J=8.8 Hz, 1H), 7.25 (m, 1H), 6.98 (d, J=8.0 Hz, 1H), 6.66 (d, J=8.8 Hz, 1H), 6.48 (s, 2H), 6.00 (s, 2H), 5.58 (s, 1H), 4.24 (s, 1H), 3.94 (s, 3H), 3.74 (s, 3H), 2.61 (m,1H), 2.5 (m, 1H), 2.42 (s, 3H), 2.34 (m, 1H), 1.98 (m, 1H). MS (ES): [M+1]⁺ calculated, C₂₇H₂₄F₂NO₆: 496.15; found, 496.65. RP-HPLC analysis (Water XTerra C18 column, 4.6×50 mm, 10-90% CH₃CN/H₂O, 10 min): retention time 3.01 min.

### Example 11

### Triflate Reduction

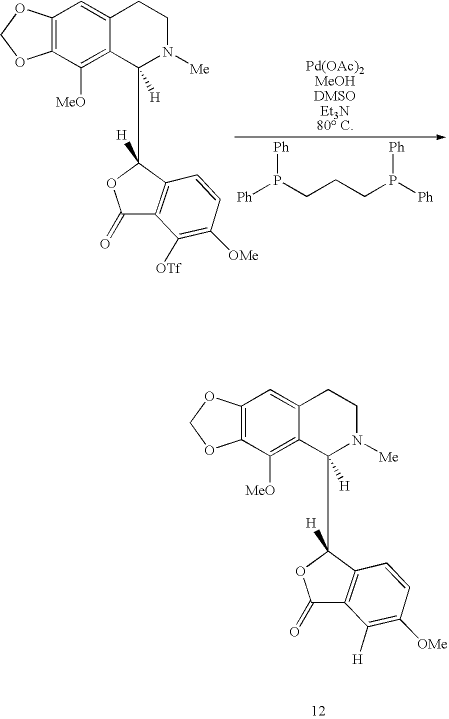

Triethylamine (2.76 mL, 19.8 mmol) was added to a mixture of the triflate (1.05 g, 1.98 mmol), palladium acetate (111 mg, 0.49 mmol), and 1,3-bis(diphenylphosphino) propane (212 mg, 0.51 mmol) in methanol (5 mL) and DMSO (5 mL). The resulting mixture was heated at 80° C. for 2 h then cooled to room temperature. The reaction mixture was diluted with ethyl acetate (300 mL) and then washed with H₂O (3×50 mL). The ethyl acetate layer was dried over Na₂SO₄, decanted, and concentrated. The crude product was purified by column chromatography (SiO₂) using 25-30% ethyl acetate-hexanes gradient to give the desired product as well as a small amount (˜4-5%) of its corresponding diastereomer (440 mg, 58%).

¹H NMR (CDCl₃, 300 MHz): δ 7.29 (d, J=2.4 Hz, 1H), 6.97 (dd, J1=8.5 Hz, J2=2.4 Hz, 1H), 6.28-6.34 (2H), 5.92-5.96 (2H), 5.67 (d, J=4.0 Hz, 1H), 4.44 (d, J=4.0 Hz, 1H), 4.04 (s, 3H), 3.84 (s, 3H), 2.51-2.67 (4H), 2.25-2.42 (2H), 1.82-1.98 (1 H). LC-Mass (ES): [M+1]⁺ calculated for C₂₁H₂₁NO₆, 384.14; found, 384.00.

### Example 12

### Sodium Hydrosulfide Addition

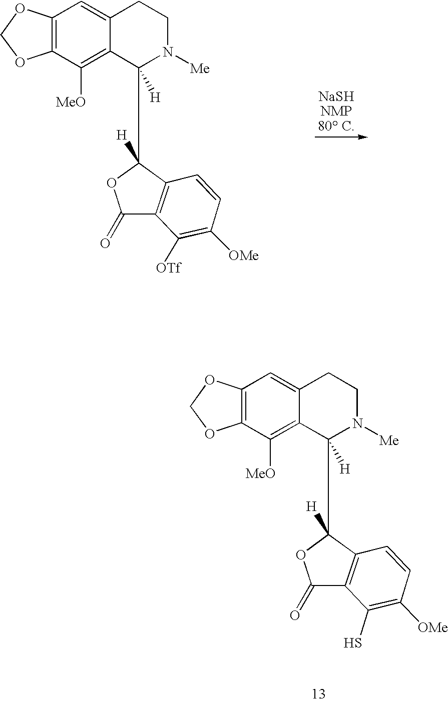

A mixture of the triflate (100 mg, 0.19 mmol) and sodium hydrosulfide hydrate (32 mg, 0.57 mmol) in 1-methyl-2-pyrrolidinone (400 μL) was sonicated until the mixture became mostly homogeneous (˜5 min). The mixture was then heated to 80° C. and stirred in a sealed vial for 1 h. The reaction mixture was cooled to room temperature and partitioned between ethyl acetate (50 mL) and H₂O (25 mL). The ethyl acetate layer was washed with saturated aq NaCl (25 mL) and then dried over Na₂SO₄. The dried product was purified by column chromatography (SiO₂) using 30-35% ethyl acetate-hexanes gradient to give compound 13 as well as a small amount (˜5%) of its corresponding diastereomer (15 mg, 19%).

¹H NMR (CDCl₃, 300 MHz): δ 6.75 (d, J=8.3 Hz, 1H), 6.25 (s, 1H), 5.95-6.10 (2H), 5.84-5.91 (2H), 5.52 (s, 1H), 4.35 (s, 1H), 3.98 (s, 3H), 3.83 (s, 3H), 2.41-2.62 (4H), 2.18-2.37 (2H), 1.78-1.95 (1H). LC-Mass (ES): [M+1]⁺ calculated for C₂₁H₂₁NO₆S, 416.11; found, 415.93.

### Example 13

### Cell cycle analysis of compounds 6a,b, 5a,b, 9,10,12, and 13

Fluorescence-activated cell sorting (FACS) analysis was used to examine the cell cycle state when the cells were incubated with compounds of formula (I) as well as several control compounds, including DMSO, noscapine (a known G2/M inhibitor), and colchicine (a known G2/M inhibitor).

In particular, HEK293 cells were cultured in DMEM media (Celigro) supplemented with 10% Fetal Bovine Serum (Tissue Culture Biologicals) and 1× Penicillin/Streptomycin/Glutamine (Gibco). The cells were grown at 37° C. with 5% CO₂, plated in six-well plates at 2.5×10⁶ cells/well, and treated for 16 to 24 hrs with the compounds at the concentrations shown in Table 1, below. The cells were then triturated off the bottom of the wells, washed 1× with PBS, then fixed with 70% ethanol, and stored at 4° C. before staining and FACS analysis.

The DNA content of cells was determined using a FacsVantage SE flow cytometry system (Becton Dickinson, San Jose, Calif.) and a Cellular DNA Flow Cytometric Analysis Kit (Roche Applied Science, Indianapolis, Ind.). Cell cycle analysis was performed using Cylchred 1.0.2 software.

When several derivatives of noscapine were examined, e.g., compounds 6a,b, a noticeably different FACS profile was observed compared to noscapine. Instead of G2/M arrested cells, the FACS profile indicated that cells treated with compounds 6a,b were accumulating in S-phase (see Table 1, below).

Additional compounds, based on the structure of compounds 6a,b, were prepared and examined by FACS, as shown below. Compounds 5a,b were observed to accumulate more cells in S-phase than 6a,b at similar concentrations. Furthermore, compounds 9a, 10, 12, and 13 were observed to have a significant accumulation of cells in G2/M (˜90%). Compound 9a was particularly potent compared to noscapine with activity being observed at 0.1 μM.

TABLE 1
HEK293 Cell Cycle of ComDounds 6a,b, 5a,b, 9a, 10, 12, and 13

| | Conc. | Percent of | Percent of | Percent of |
|---|---|---|---|---|
| Drug | (μM) | Cells in G1 | Cells in S | Cells in G2/M |
| DMSO | 0.5% | 484 | 38.4 | 13.2 |
| colchicine | 10 | 2.7 | 13.9 | 83.4 |
| noscapine | 50 | 19.2 | 31.1 | 49.6 |
| 6a,b | 50 | 12.1 | 71.2 | 16.7 |
| 5a,b | 50 | 13.1 | 80.7 | 6.3 |
| 9a | 0.1 | 1 | 6.9 | 92.1 |
| 10 | 50 | 2 | 8.8 | 89.2 |
| 12 | 1 | 3.8 | 6.4 | 89.8 |
| 13 | 5 | 3.2 | 7.2 | 89.6 |

### Example 14

### Cell Cycle Analysis of 5a,b on Phytohemagglutinin-stimulated and Non-stimulated Peripheral Blood Lymphocytes

The effects of compounds 5a,b on the cell cycle were further examined on phytohemagglutinin (PHA)-stimulated and non-stimulated peripheral blood lymphocytes (PBLs).

PBLs were cultured in DMEM media (Celigro) supplemented with 10% Fetal Bovine Serum (Tissue Culture Biologicals) and 1× Penicillin/Streptomycin/Glutamine (Gibco). The cells were grown at 37° C. with 5% CO₂, plated in six-well plates at 2.5-5×10⁶ cells/well, and treated for 16 to 24 hrs with the compounds at the concentrations shown in Table 2, below, with or without 1 μg/ml PHA (Sigma). The cells were then triturated off the bottom of the wells, washed 1× with PBS, then fixed with 70% ethanol, and stored at 4° C. before staining and FACS analysis.

TABLE 2
Cell Cycle Data for 5a,b with PBL Cells

| | | | | | Percent |
|---|---|---|---|---|---|
| | | Conc. | Percent of | Percent of | of Cells |
| Cell Line | Drug | (μM) | Cells in G1 | Cells in S | in G2/M |
| PBL | DMSO | 0.5% | 96.3 | 1.7 | 2 |
| PBL | 5a,b | 25 | 91.6 | 6.8 | 1.5 |
| PHA-stim. PBL | DMSO | 0.5% | 80.5 | 15.6 | 3.9 |
| PHA-stim. PBL | 5a,b | 25 | 69.3 | 25.3 | 5.4 |

As shown in Table 2, there was little effect on non-stimulated PBLs. When PBLs were stimulated by PHA to undergo cell division, there was an increase in cells present in S-phase and G2/M compared to non-stimulated PBLs. Furthermore, PHA-stimulated PBLs had a greater accumulation of cells present in S-phase versus the DMSO control (25.3% vs. 15.6%).

### Example 15

### Effect of Compounds 5a, 5a,b, and 9a on Cell Viability

The effect of various doses of compounds 5a, 5a,b, and 9a on the viability of HEK293 cells was examined using the CellTiter 96 Aqueous One Solution Cell Proliferation assay (Promega).

The HEK293 cells were cultured in DMEM media (Cellgro) or phenol red-free DMEM (Gibco) supplemented with 10% Fetal Bovine Serum (Tissue Culture Biologicals) and 1× Penicillin/Streptomycin/Glutamine (Gibco). The cells were grown at 37° C. with 5% CO₂.

HEK293 cells were seeded into each well of a 96-well plate (5000 cells/well) and incubated overnight to allow the cells to attach. The culture media was removed and replaced with 100 μl of phenol red-free media containing the compounds 5a, 5a,b, and 9a at various concentrations. The cells were allowed to grow in the presence of the compounds for 48 hrs. At the end of the treatment, 20 μl of CellTiter 96 Aqueous One Solution reagent was added directly into each well, and the cells were incubated at 37° C. for 2 hrs. This reagent converts to a form that absorbs light at 490 nm by living cells. Thus, absorbance at 490 nm is proportional to the number of viable cells. The 490 nm absorbance was measured on a BioRad plate reader. Control wells containing no cells were included in this assay to correct for background absorbance.

As Table 3 shows, all three compounds 5a, 5a,b, and 9a have EC₅₀ values below 1 μM in this assay. Compound 9a was especially potent with an EC₅₀ below 50 nM that is similar to results obtained with the anticancer agent, paclitaxel.

TABLE 3
Effects of Compounds 5a,b, 5a, and 9a on HEK293 Cell Viability

| Drug | EC₅₀ (nM) |
|---|---|
| Noscapine | ˜10,000 |
| Nocodazole | <20 |
| Paclitaxel | <100 |
| Compound 5a,b | 400 |
| Compound 5a | 300 |
| Compound 9a | <50 |

### Example 16

### Effect of Compound 9a on the Viability of Cancer Cells

The effect of compound 9a on the viability of cancer cell lines as well as dividing and non-dividing mouse Swiss3T3 cells was studied.

In particular, the cell proliferation assay, as described in Example 15, was used to test the effect of a 48 to 72 hr exposure of these cell lines to compound 9a at various concentrations. As Table 4 indicates, compound 9a was found to have activity against a broad range of cancer cell lines. However, no effect was observed with compound 9a even at 50 μM with the non-dividing Swiss 3T3 fibroblasts.

TABLE 4
Effects of Compound 9a on the Viability of Cancer Cell Lines

| Cell Line | Tissue | EC₅₀ (nm) |
|---|---|---|
| H4 | glioma | <50 |
| HeLa | cervical adenocarcinoma | <50 |
| U937 | lymphoma | <50 |
| HL-60 | leukemia | <50 |
| NCl/ADR | breast adenocarcinoma | <50 |
| A549 | non-small cell lung carcinoma | <100 |
| HT29 | colorectal adenocarcinoma | <100 |
| PC3 | prostate adenocarcinoma | <100 |
| HCT15 | colorectal adenocarcinoma | <100 |
| SW480 | colorectal adenocarcinoma | <100 |
| HOP-18 | non-small cell lung carcinoma | <100 |
| OVCAR-4 | ovarian adenocarcinoma | <100 |
| Swiss 3T3 | dividing fibroblast | <100 |
| Swiss 3T3 | non-dividing fibroblast | >50,000 |

### Example 17

### Topoisomerase Assays for Noscapine Derivatives

The ability of compounds that cause S-phase cell cycle arrest to inhibit topoisomerase I and topoisomerase II was examined. Analysis of topoisomerase activity was performed in 20 μl reactions with either topoisomerase I reaction buffer (10 mM TRIS-HCl, pH 7.5; 100 mM NaCl; 1 mM 2-mercaptoethanol) or topoisomerase II reaction buffer (50 mM Tris-HCl, pH 8; 120 mM KCl; 10 mM MgCl₂; 0.5 mM dithiothreitol; 0.5 mM ATP; 30 μg BSA/ml) containing 125 ng supercoiled pBluescript DNA and either 2 U topoisomerase I (Sigma-Aldrich) or topoisomerase II (Topogen Inc.). After reaction at 37° C. for 20 minutes, the reactions were terminated through the addition of sodium dodecyl sulfate to 1% w/v and heating to 65° C. for 10 minutes. Proteinase K was then added to 10 μg/ml and digestion was allowed to proceed at 37° C. for 20 minutes before electrophoresis of the DNA in 0.8% agarose gels containing 0.5 μg/ml ethidium bromide for 18 hours at 75V in 0.5×TBE buffer. DNA was then visualized using a UV-transilluminator and the image was captured using a BioRad CCD camera and gel documentation system.

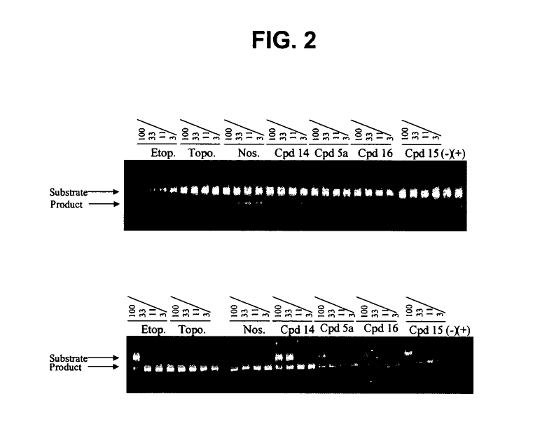

As shown in the FIG. 2, the known topoisomerase I inhibitor topotecan blocked this enzymatic activity at concentrations below 3 μM in vitro. While neither etoposide nor noscapine inhibited of topoisomerase I activity, compounds 14 and 15 inhibited this enzyme at concentrations greater than 33 μM. Compounds 5a and 16 inhibited topoisomerase I activity at concentrations greater than 100 μM.

The known compound etoposide inhibited the activity of topoisomerase II at concentrations greater than 33 μM (FIG. 2), while neither topotecan nor noscapine acts as inhibitors. Compound 14 inhibited topoisomerase II activity at concentrations greater than 33 μM, and compounds 15, 5a, and 16 inhibited topoisomerase II activity at concentrations greater than 100 μM.

### Example 18

### Effect of Compound 9a on Microtubule Polymerization

Microtubule polymerization assays were performed to determine if compounds, such as compound 9a, cause G2/M cell cycle arrest by affecting microtubule dynamics. A microtubule polymerization assay was performed that takes advantage of the observation that there is an increase in turbidity that can be measured at 340 nm as tubulin heterodimers polymerize to form microtubules. In brief, HTS-tubulin (90% tubulin plus 10% microtubule associated proteins; Cytoskeleton Inc., Denver, Colo.) was resuspended in G-PEM buffer (80 mM Pipes, pH 6.9; 2 mM MgCl₂; 0.5 mM EDTA; 1 mM GTP) to 10 mg/ml. This suspension was clarified by centrifugation for 10 min. at 225,000×g. The supernatant was then diluted with G-PEM buffer to 2 mg/ml and 100 μl of this solution was added to the appropriate number of ½-area wells in a 96-well format (Corning). Each compound (10 μl of a 10× final concentration in 10% DMSO) was added to the wells in duplicate and immediately put into a pre-warmed (37° C.) Spectromax 190 plate reader. The absorbance was read at 340 nm immediately (time=0) and every minute thereafter for 30 minutes.

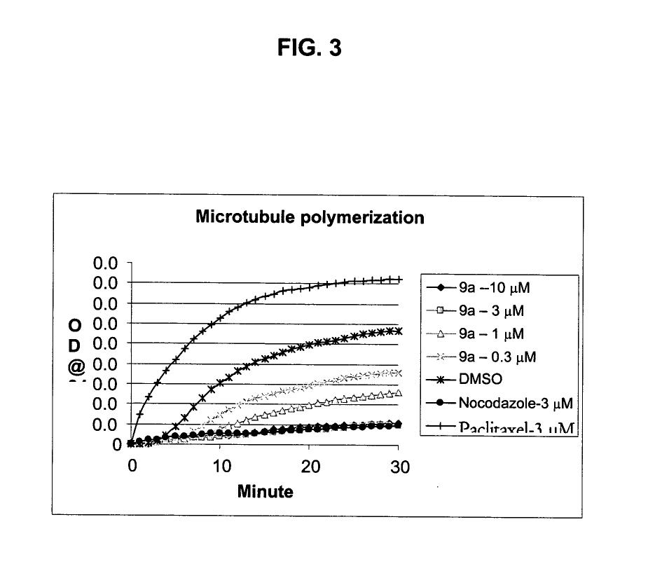

The tubulin mix was incubated in the presence of paclitaxel (a microtubule stabilizing agent), nocodazole (a microtubule destabilizing agent), vehicle control (DMSO), or varying concentrations of compound 9a (FIG. 3). As expected, paclitaxel increased microtubule polymerization and nocodazole decreased microtubule polymerization. Compound 9a caused a dose-dependent decrease of microtubule polymerization, thereby establishing this compound as a microtubule-destabilizing drug.

The foregoing embodiments and advantages are merely exemplary and are not to be construed as limiting the present invention. The description of the present invention is intended to be illustrative, and not to limit the scope of the claims. Many alternatives, modifications, and variations will be apparent to those skilled in the art.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

1. A compound of formula (I): 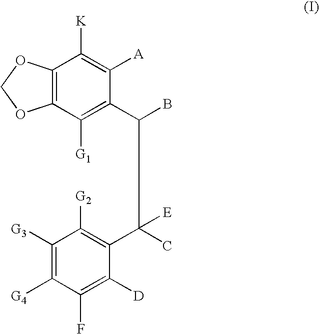
wherein:
A is 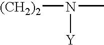
and forms a nitrogen-containing heterocycloalkyl ring with B,
in which Y is:
(a) hydrogen, C₁₋₆ alkyl, or C₁₋₆ alkylaryl,
(b) —C(O)—C₁₋₆ alkyl or —C(O)—C₁₋₆ alkylaryl,
(c) —CH₂—CH(OH)—CH₂-Z, where Z is C₁₋₆ alkyl or —O—C -6 alkyl,
(d) aryl, or
(e) heteroaryl;
B is —OH, halogen, or a single bond that forms a six-membered heterocycloalkyl ring with A;
C is hydrogen, C₁₋₆ alkyl, or halogen;
D is (i) —CH₂-halogen, —CH(O), —COOH, —C(O)—O—C₁₋₆ alkyl, —C(O)—O—C₁₋₆ alkylaryl, —CH₂OH, or —(CH₂)ₙ—CH₃, wherein n is 1, 2, or 3, or
(ii) together with E forms a five- or six-membered cycloalkyl or heterocycloalkyl ring;
E is —OH or C₁₋₆ alkyl, or together with D forms a five- or six-membered cycloalkyl or heterocycloalkyl ring, wherein this heterocycloalkyl ring contains —C(O)O—, —C(O)NH—, —C(S)O—, or —C(S)NH—;
F is hydrogen, —O—C₁₋₆ alkyl, —O—C₁₋₆ alkylaryl, —O—C₁₆ alkylheteroaryl, halogen, aryl, C₁₋₆—SH, thio-C₁₋₆ alkyl, —S-aryl, —O—SO₂—C₁₋₆alkyl, —O—SO₂—C₁₋₆ alkylaryl, cyano, or NR1R2, where R1 and R2 are independently hydrogen, C₁₋₆ alkyl, C₁₋₆ alkylaryl, cyano, aryl, heteroaryl, —SO₂—C₁₋₆alkyl, or —SO₂—N(C₁₋₆alkyl)(C₁₋₆alkyl);
G₁ to G₄ independently represent hydrogen, aryl, halogen, C₁₋₆ alkyl, hydroxyl, —S—C₁₋₆ alkyl, nitro, —O—C₁₋₆ alkyl, —O—C₁₋₆ alkylaryl, or —(CH₂)ₓNR1R2, where x is 0, 1, or 2 and where R1 and R2 are independently hydrogen, C₁₋₆alkyl, C₁₋₆alkylaryl, cyano, aryl, heteroaryl, or acyl, or two adjacent G₂ to G₄ groups together comprise an alkylene —(CH₂)ₘ—, where m is 3 or 4, to form a cycloalkyl ring, or together comprise an alkylene dioxy —O—(CH₂)ₙ—O—, where n is 1, 2, or 3, to form a heterocycloalkyl ring; and
K is C₁₋₆alkyl, halogen, cyano, aryl, hydrogen, hydroxyl, thio-C₁₋₆alkyl, sulfonyl, sulfoxyl, nitro, —O—C₁₋₆alkyl, —O—C₁₋₆alkylaryl, or NR1R2, where R1 and R2 are independently hydrogen, C₁₋₆alkyl, C₁₋₆alkylaryl, cyano, aryl, heteroaryl, or acyl;
wherein one or more of said alkyl, aryl, heteroaryl, cycloalkyl, heterocycloalkyl, and alkylaryl groups ate optionally substituted with one or more suitable substituents; a salt thereof, a solvate thereof, a solvated salt thereof, or a combination of two or more thereof;
provided that when A is —(CH₂)₂—N(Y)— and forms a nitrogen-containing heterocycloalkyl ring with B, and D together with E forms an unsubstituted five-membered heterocycloalkyl ring that contains —C(O)O—, then:
(i) F is not unsubstituted —O—C₁₋₆alkyl or dialkylamino-substituted —O—C₁₋₆alkyl when G₁ is hydrogen, hydroxyl, or unsubstituted —O—C₁₋₆alkyl, G₂is hydrogen, halogen, or a nitrogen-containing radical, G₃is hydrogen, G₄is hydroxyl or unsubstituted —O—C₁₋₆alkyl, and Y is hydrogen, unsubstituted C₁₋₆alkyl, oxo-substituted C₁₋₆alkyl, thiocarbamoyl-substituted C₁₋₆alkyl, hydroxy-substituted C₁₋₆alkyl, or heteroaryl,
(ii) F is not —NO₂ or NR1R2 where R1 and R2 are both hydrogen or the same oxo-substituted C₁₋₆alkyl (a) when at least three of G₁, G₂, G₃, and G₄ are the same unsubstituted —O—C₁₋₆alkyl or (b) when G₂ is —NO₂, and
(iii) F is not hydrogen (a) when G₂, G₃, and G₄ are all hydrogen or (b) when G₂ and G₃ or G₃ and G₄ together comprise a methylenedioxy or (c) when at least two of G₂, G₃, and G₄ are unsubstituted —O—C₁₋₆ alkyl or (d) when G₁ is unsubstituted —O—C₁₋₆alkyl and G₄ is a nitrogen-containing radical or halogen.

2. The compound of claim 1, wherein A is —(CH₂)₂—N(Y)— and forms a nitrogen-containing heterocycloalkyl ring with B.

3. The compound of claim 2, wherein Y is hydrogen, C₁₋₆alkyl, or C₁₋₆alkylaryl.

4. The compound of claim 1, wherein D together with E forms a substituted or unsubstituted five- or six-membered heterocycloalkyl ring that contains —C(O)O—, —C(O)NH—, —C(S)O—, or —C(S)NH—.

5. The compound of claim 1, wherein D together with E forms a five-membered heterocycloalkyl ring that contains —C(O)O—.

6. The compound of claim 1, wherein A is —(CH₂)₂—N(Y)— and forms a nitrogen-containing heterocycloalkyl ring with B, and D together with E forms a substituted or unsubstituted five- or six-membered heterocycloalkyl ring that contains —C(O)O—, —C(O)NH—, —C(S)O—, or —C(S)NH—.

7. The compound of claim 1, wherein A is —(CH₂)₂—N(Y)— and forms a nitrogen-containing heterocycloalkyl ring with B, and D together with E forms a five-membered heterocycloalkyl ring that contains —C(O)O—.

8. The compound of claim 6 or 7, wherein Y is hydrogen, C₁₋₆alkyl, or C₁₋₆alkylaryl.

9. The compound of claim 1, 6, or 7, wherein K is hydrogen.

10. The compound of claim 1, 6, or 7, wherein G₁ to G₄ each independently represents hydrogen or —O—C₁₋₆alkyl.

11. The compound of claim 6 or 7, wherein said compound is present as a racemic mixture.

12. The compound of claim 11, wherein one isomer of said compound is present in an amount greater than 50% of said racemic mixture.

13. The compound of claim 11, wherein one isomer of said compound is present in an amount greater than 75% of said racemic mixture.

14. The compound of claim 11, wherein one isomer of said compound is present in an amount greater than 90% of said racemic mixture.

15. A pharmaceutical composition comprising a pharmaceutically effective amount of a compound of claim 1 and a pharmaceutically acceptable carrier.

16-120. (Cancelled)

121. A method of making the compound of claim 1 by direct nucleophilic substitution, comprising reacting a compound of formula (IV): 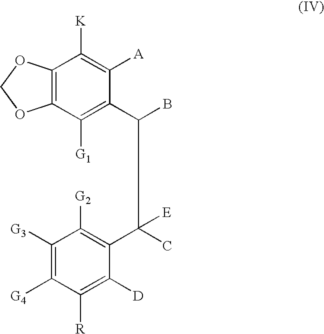
wherein each of the variables other than R are defined as in claim 1 and R is a suitable leaving group, with a suitable nucleophile to form a compound according to formula (I).

122. The method of claim 121, wherein R is a halogen, —O—C₁₋₆alkyl or —O—SO₂—C₁₋₆alkyl.

123. The method of claim 120, wherein said compound of formula (IV) is mixed with a suitable catalyst.

124. The method of claim 123, wherein said suitable catalyst comprises tris(dibenzylideneacetone)-dipalladium chloroform adduct, 1,1′-bis(diphenylphosphino)ferrocene DPPF), tetrakis(triphenylphosphine)palladium or mixtures thereof.

125. The method of claim 123, wherein a suitable base is added to the mixture of said compound of formula (IV) and said suitable catalyst.

126. A method of making the compound of claim 1 by direct alkylation, comprising reacting a compound of formula (VI): 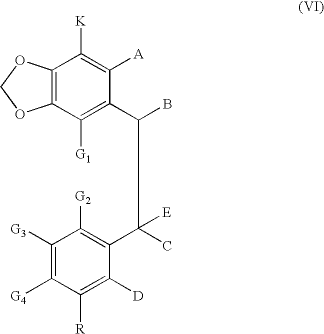
wherein each of the variables other than R are defined as in claim 1 and R is a suitable leaving group, with a suitable donor to form a compound according to formula (I).

127. The method of claim 126, wherein R is a hydroxyl or substituted hydroxyl.

128. The method of claim 126, wherein said suitable donor is C₁₋₆alkyl halide or substituted C₁₋₆alkyl halide.

129. The method of claim 126, wherein said compound of formula (VI) is mixed with a suitable catalyst.

130. The method of claim 129, wherein said suitable catalyst comprises tetrabutylammonium iodide.

131. A method of making the compound of claim 1 by alkoxide addition, comprising reacting a compound of formula (X): 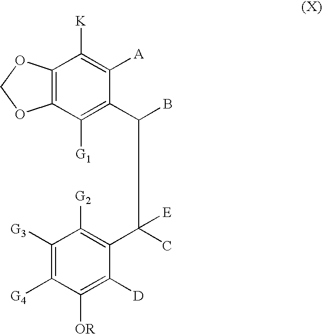
wherein each of the variables other than R are defined as in claim 1 and R is C₁₋₆alkyl, with a base in a suitable solvent to form an alkoxide, and reacting the alkoxide with an electrophilic alkylating agent to form a compound according to formula (I).

132. The method of claim 131, wherein said suitable solvent comprises toluene, 1-methyl-2-pyrrolidinone or mixtures thereof.

133. The method of claim 131, wherein the molar ratio of said compound of formula (X) to said electrophilic alkylating agent is about 1:1 to about 1:10.

134. The method of claim 131, wherein the molar ratio of said compound of formula (X) to said electrophilic alkylating agent is about 1:1 to about 1:3.

135. The method of claim 131, wherein said electrophilic alkylating agent is an alkyl halide or heteroaryl.

136. A method of making the compound of claim 1, comprising converting a compound of formula (II): 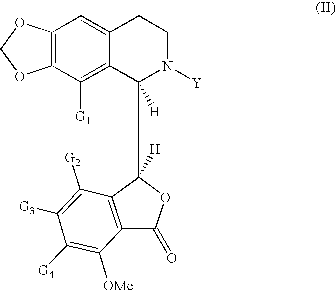
wherein:
Y is:
(a) hydrogen, C₁₋₆alkyl, or C₁₋₆alkylaryl,
(b) —C(O)—C₁₋₆alkyl or —C(O)—C₁₋₆alkylaryl,
(c) —CH₂—CH(OH)—CH₂-Z, where Z is C₁₋₆alkyl or —O—C₁₋₆alkyl,
(d) aryl, or
(e) heteroaryl; and
G₁ to G₄ independently represent hydrogen, aryl, halogen, C₁₋₆alkyl, hydroxyl, —S—C₁₋₆alkyl, nitro, —O—C₁₋₆alkyl, —O—C₁₋₆alkylaryl, or —(CH₂)ₓNR1R2, where x is 0, 1, or 2 and where R1 and R2 are independently hydrogen, C₁₋₆alkyl, C₁₋₆alkylaryl, cyano, aryl, heteroaryl, or acyl, or
two adjacent G₂ to G₄ groups together comprise an alkylene —(CH₂)ₘ—, where m is 3 or 4, to form a cycloalkyl ring, or together comprise an alkylene dioxy —O—(CH₂)ₙ—O—, where n is 1, 2, or 3, to form a heterocycloalkyl ring;
a salt thereof, a solvate thereof, a solvated salt thereof, or a combination of two or more thereof;
into a single stereoisomer of formula (III): 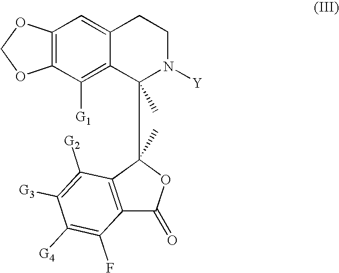
wherein G₁, G₂, G₃, G₄, and Y are as defined above, and F is —O—C₂₋₆alkyl, —O—C₁₋₆alkylaryl, —O—C₁₋₆alkylheteroaryl, halogen, aryl, C₁₋₆alkyl, —SH, thio-C₁₋₆alkyl, —S-aryl, —O—SO₂—C₁₋₆alkyl, —O—SO₂—C₁₋₆alkylaryl, cyano, or NR1R2, where R1 and R2 are independently hydrogen, C₁₋₆alkyl, C₁₋₆alkylaryl, cyano, aryl, heteroaryl, —SO₂—C₁₋₆alkyl, or —SO₂—N(C₁₋₆alkyl)(C₁₋₆alkyl), provided that F is not —O-t-C₄H₉ or —O—CH₂CH₂N(C₂H₅)₂;
wherein one or more of said alkyl, aryl, heteroaryl, and alkylaryl groups are optionally substituted with one or more suitable substituents;
a salt thereof, a solvate thereof, a solvated salt thereof, or a combination of two or more thereof.
